(19) United States
(12) Patent Application Publication
(10) Pub. No.: US 2025/0244299 A1
(43) Pub. Date: Jul. 31, 2025

(54) CHARACTERIZATION OF BINDING-RELATED CQAS IN THEREAPEUTIC MABS

(71) Applicant: Regeneron Pharmaceuticals, Inc., Tarrytown, NY (US)

(72) Inventors: Yuetian Yan, Chappaqua, NY (US); Tao Xing, White Plains, NY (US); Shunhai Wang, Scarsdale, NY (US); Ning Li, New Canaan, CT (US)

(21) Appl. No.: 19/035,461

(22) Filed: Jan. 23, 2025

Related U.S. Application Data
(60) Provisional application No. 63/625,511, filed on Jan. 26, 2024.

(51) Int. Cl. G01N 30/72; C12Q 1/37; G01N 30/02; G01N 30/06; G01N 30/88; G01N 33/68

(52) U.S. Cl. CPC G01N 30/7233; C12Q 1/37; G01N 33/6848; G01N 33/6857; G01N 2030/027; G01N 2030/067; G01N 2030/8831; G01N 2333/70535; G01N 2333/96466

(57) ABSTRACT

The present disclosure generally pertains to methods for identifying the binding of a peptide or protein to a binding partner using affinity-resolved size exclusion chromatography coupled to mass spectrometry.

### CROSS REFERENCE TO RELATED APPLICATION

The present application claims priority to and the benefit of U.S. Patent Application No. 63/625,511, entitled “CHARACTERIZATION OF BINDING-RELATED CQAS IN THERAPEUTIC MABS,” which was filed on Jan. 26, 2024, the disclosure of which is hereby incorporated by reference in its entirety.

### BACKGROUND

Assessment of critical quality attributes (CQAs) is an important aspect during the development of therapeutic monoclonal antibodies (mAbs). Attributes that affect either the target binding or Fc receptor engagement may have direct impacts on the drug safety and efficacy, and thus are considered as CQAs.

Therapeutic monoclonal antibodies (mAbs) often host a large number of post-translational modifications (PTMs), which can be either intended or unintended. These modifications, such as glycosylation, deamidation, and oxidation, are a result of various mechanisms during the mAb production, manufacturing, and storage. Of these modifications, some have little or no impact on product quality, and thus are not considered as critical. Others may impair the efficacy or safety of the drug products and are therefore defined as CQAs. Additionally, modifications to the Fc region of the mAb may enhance the mAb:receptor interaction, thus improving the pharmacokinetic performances of mAbs. Understanding the modifications within these regions are potentially critical and worth thorough evaluations during drug development.

It will be appreciated that a need exists for methods to identify and/or characterize mAb-receptor binding to understand the impact the modification has on mAb function.

### SUMMARY

The present disclosure provides methods of characterizing the binding of a protein to an antigen, the methods comprising:
incubating the antigen with a sample comprising the protein to form a mixture comprising protein-antigen complex and protein that is not bound to the antigen;
separating the mixture on a liquid chromatography column to separate the protein-antigen complex from the protein that is not bound to the antigen and to form an eluate comprising the protein-antigen complex and the protein that is not bound to the antigen;
contacting the eluate with a denaturation solvent to form a denatured sample;
determining a mass of the protein-antigen complex and a mass of the protein that is not bound to the antigen with a mass spectrometer that is coupled to the liquid chromatography column; and
analyzing the mass of the protein-antigen complex and the mass of the protein that is not bound to the antigen to characterize the binding of the protein to the antigen.

The present disclosure provides methods for analyzing the effect of glycosylation on the binding of a protein to an antigen, the methods comprising:
incubating the antigen with a sample comprising the protein to form a mixture comprising protein-antigen complex and protein that is not bound to the antigen;
separating the mixture on a liquid chromatography column to separate the protein-antigen complex from the protein that is not bound to the antigen and to form an eluate comprising the protein-antigen complex and the protein that is not bound to the antigen;
contacting the eluate with a denaturation solvent to form a denatured sample;
determining a mass of the protein-antigen complex and a mass of the protein that is not bound to the antigen with a mass spectrometer that is coupled to the liquid chromatography column; and
analyzing the mass of the protein-antigen complex and the mass of the protein that is not bound to the antigen to analyze the effect of glycosylation on the binding of the protein to the antigen.

The present disclosure provides methods of characterizing the binding of a protein to an antigen, the methods comprising:
incubating the antigen with a sample comprising the protein to form a mixture comprising protein-antigen complex and protein that is not bound to the antigen;
separating the mixture on a liquid chromatography column to separate the protein-antigen complex from the protein that is not bound to the antigen and to form an eluate comprising the protein-antigen complex and the protein that is not bound to the antigen;
contacting the eluate with a denaturation solvent to form a denatured sample;
determining a mass of the protein-antigen complex and a mass of the protein that is not bound to the antigen with a mass spectrometer that is coupled to the liquid chromatography column;
analyzing the mass of the protein-antigen complex and the mass of the protein that is not bound to the antigen to characterize the binding of the protein to the antigen;
separating the protein that is not bound to the antigen on a strong cation exchange (SCX) chromatography column to separate modified protein and unmodified protein and to form an eluate comprising modified and unmodified protein;
contacting the eluate comprising the modified protein and the unmodified protein with a denaturation solvent to form a denatured sample;
determining a mass of the modified protein and a mass of the unmodified protein with a mass spectrometer that is coupled to the strong cation exchange chromatography column; and
analyzing the mass of the modified protein and the mass of the unmodified protein to determine if the protein that is not bound to the antigen has a molecular modification.

This disclosure provides methods identifying the binding of a peptide or protein to a binding partner. In some exemplary embodiments, the methods can comprise: contacting a sample comprising the peptide or protein with a binding partner to form a complex, contacting the complex to a liquid chromatography column to separate bound and free material in an eluate, contacting the eluate to a denaturation solvent to form a denatured sample, contacting the denatured sample to a mass spectrometer that is coupled to the liquid chromatography column to determine a mass of the bound and free material, and analyzing the mass of the bound and free material to identify the binding of the peptide or protein to the binding partner.

In one aspect, the peptide or protein is digested prior to contacting the sample with a binding partner.

In one aspect, the digestive enzyme is selected from a group consisting of pepsin, trypsin, Tryp-N, chymotrypsin, Lys-N, Lys-C, Asp-N, Arg-C, Glu-C, papain, IdeS, IdeZ, IgdE, glyserias, and variants or combinations thereof.

In one aspect, the digested sample is partially reduced to form a digested and partially reduced sample. In a specific aspect, the reducing agent is dithiothreitol (DTT), ß-mercaptoethanol, Ellman's reagent, hydroxylamine hydrochloride, sodium cyanoborohydride, tris(2-carboxyethyl)phosphine hydrochloride (TCEP-HCl), or combinations thereof.

In one aspect, the binding partner is a receptor to the peptide or protein.

In one aspect, the ratio of receptor to peptide or protein is between 1:10 and 10:1. In a specific aspect, the ratio of receptor to peptide or protein is 1:4, 1:2, 1:1, 2:1, or 4:1.

In one aspect, the binding partner is an antigen to the peptide or protein.

In one aspect, wherein the ratio of the antigen to peptide or protein is between 1:10 and 10:1. In a specific aspect, the ratio of antigen to peptide or protein is 1:2, 2:3, 1:1, 2:1, 3:2, 4:1, or 5:2

In one aspect, the protein of interest is selected from a group comprising an antibody, a monoclonal antibody, a bispecific antibody, an antibody fragment, an antibody-derived protein, an antigen-binding protein, an antibody-drug conjugate, or a fusion protein.

In one aspect, the liquid chromatography comprises reversed phase liquid chromatography, ion exchange chromatography, anion exchange chromatography, weak cation exchange chromatography, strong cation exchange chromatography, size exclusion chromatography, affinity chromatography, hydrophobic interaction chromatography, hydrophilic interaction liquid chromatography (HILIC), mixed-mode chromatography, or a combination thereof. In a specific aspect, the liquid chromatography comprises size exclusion chromatography. In another specific aspect, the mobile phase of the liquid chromatography is ammonium acetate.

In one aspect, the denaturation solvent comprises acetonitrile (ACN), water and formic acid (FA). In a specific aspect, the denaturation solvent is added at a 1:1 ratio with the eluent.

In one aspect, a splitter is used to connect the chromatography column and the mass spectrometer.

In one aspect, the splitter is used after denaturation to divert a low flow to the mass spectrometer and a high flow to a UV detector.

In one aspect, the binding partner of the at least one peptide or protein is protein A, protein G, Fcγ receptor, FcγRIIIa, anti-human Fc antibody, neonatal Fc receptor, Fc epsilon RI, anti-idiotype antibody, an antigen or complement component C1q.

In one aspect, the mass spectrometer is an electrospray ionization mass spectrometer, nano-electrospray ionization mass spectrometer, a triple quadrupole mass spectrometer, a quadrupole mass spectrometer, or an ultra-high mass range hybrid quadrupole mass spectrometer. In a specific aspect, the mass spectrometer comprises a nano-electrospray ionization-mass spectrometer.

This disclosure further provides methods for identifying or characterizing the effect of glycosylation on the binding of a peptide or protein to a binding partner. In some exemplary embodiments, the methods can comprise: contacting a sample comprising the peptide or protein with a binding partner to form a complex, contacting the complex to a liquid chromatography column to separate bound and free material in an eluate, contacting the eluate to a denaturation solvent to form a denatured sample, contacting the denatured sample to a mass spectrometer that is coupled to the liquid chromatography column to determine a mass of the bound and free material, and analyzing the mass of the bound and free material to identify the binding of the peptide or protein to the binding partner.

In one aspect, the peptide or protein is digested prior to contacting the sample with a binding partner.

In one aspect, the digestive enzyme is selected from a group consisting of pepsin, trypsin, Tryp-N, chymotrypsin, Lys-N, Lys-C, Asp-N, Arg-C, Glu-C, papain, IdeS, and variants or combinations thereof. In another specific aspect, the digestive enzyme is IdeS.

In one aspect, the digested sample is partially reduced to form a digested and partially reduced sample. In a specific aspect, the reducing agent is dithiothreitol (DTT), 8-mercaptoethanol, Ellman's reagent, hydroxylamine hydrochloride, sodium cyanoborohydride, tris(2-carboxyethyl)phosphine hydrochloride (TCEP-HCl), or combinations thereof.

In one aspect, the binding partner is a receptor to the peptide or protein.

In one aspect, the ratio of receptor to peptide or protein is between 1:10 and 10:1. In a specific aspect, the ratio of receptor to peptide or protein is 1:4, 1:2, 1:1, 2:1, or 4:1.

In one aspect, the binding partner is an antigen to the peptide or protein.

In one aspect, wherein the ratio of the antigen to peptide or protein is between 1:10 and 10:1. In a specific aspect, the ratio of antigen to peptide or protein is 1:2, 2:3, 1:1, 2:1, 3:2, 4:1, or 5:2.

In one aspect, the protein of interest is selected from a group comprising an antibody, a monoclonal antibody, a bispecific antibody, an antibody fragment, an antibody-derived protein, an antigen-binding protein, an antibody-drug conjugate, or a fusion protein.

In one aspect, the liquid chromatography comprises reversed phase liquid chromatography, ion exchange chromatography, anion exchange chromatography, weak cation exchange chromatography, strong cation exchange chromatography, size exclusion chromatography, affinity chromatography, hydrophobic interaction chromatography, hydrophilic interaction liquid chromatography (HILIC), mixed-mode chromatography, or a combination thereof. In a specific aspect, the liquid chromatography comprises size exclusion chromatography. In another specific aspect, the mobile phase of the liquid chromatography is ammonium acetate.

In one aspect, the denaturation solvent comprises acetonitrile (ACN), water and formic acid (FA). In a specific aspect, the denaturation solvent is added at a 1:1 ratio with the eluent.

In one aspect, a splitter is used to connect the chromatography column and the mass spectrometer.

In one aspect, the splitter is used after denaturation to divert a low flow to the mass spectrometer and a high flow to a UV detector.

In one aspect, the binding partner of the at least one peptide or protein is protein A, protein G, Fcγ receptor, FcγRIIIa, anti-human Fc antibody, neonatal Fc receptor, Fc epsilon RI, anti-idiotype antibody, an antigen or complement component C1q.

In one aspect, the mass spectrometer is an electrospray ionization mass spectrometer, nano-electrospray ionization mass spectrometer, a triple quadrupole mass spectrometer, a quadrupole mass spectrometer, or an ultra-high mass range hybrid quadrupole mass spectrometer. In a specific aspect, the mass spectrometer comprises a nano-electrospray ionization-mass spectrometer.

This disclosure further provides methods for identifying or characterizing the effect of an Fc region variant on the binding of a peptide or protein to a binding partner. In some exemplary embodiments, the methods can comprise: contacting a sample comprising the peptide or protein with a binding partner to form a complex, contacting the complex to a liquid chromatography column to separate bound and free material in an eluate, contacting the eluate to a denaturation solvent to form a denatured sample, contacting the denatured sample to a mass spectrometer that is coupled to the liquid chromatography column to determine a mass of the bound and free material, and analyzing the mass of the bound and free material to identify the binding of the peptide or protein to the binding partner.

In one aspect, the binding partner is a receptor to the peptide or protein.

In one aspect, the ratio of receptor to peptide or protein is between 1:10 and 10:1. In a specific aspect, the ratio of receptor to peptide or protein is 1:6, 1:3, 2:3, 4:3, and 6:3.

In one aspect, the protein of interest is selected from a group comprising an antibody, a monoclonal antibody, a bispecific antibody, an antibody fragment, an antibody-derived protein, an antigen-binding protein, an antibody-drug conjugate, or a fusion protein.

In one aspect, the liquid chromatography comprises reversed phase liquid chromatography, ion exchange chromatography, anion exchange chromatography, weak cation exchange chromatography, strong cation exchange chromatography, size exclusion chromatography, affinity chromatography, hydrophobic interaction chromatography, hydrophilic interaction liquid chromatography (HILIC), mixed-mode chromatography, or a combination thereof. In a specific aspect, the liquid chromatography comprises size exclusion chromatography. In another specific aspect, the mobile phase of the liquid chromatography is ammonium acetate.

In one aspect, the denaturation solvent comprises acetonitrile (ACN) and water. In a specific aspect, the denaturation solvent is added at a 1:1 ratio with the eluent.

In one aspect, a splitter is used to connect the chromatography column and the mass spectrometer.

In one aspect, the splitter is used after denaturation to divert a low flow to the mass spectrometer and a high flow to a UV detector.

In one aspect, the binding partner of the at least one peptide or protein is protein A, protein G, Fcγ receptor, FcγRIIIa, anti-human Fc antibody, neonatal Fc receptor, Fc epsilon RI, anti-idiotype antibody, an antigen or complement component C1q.

In one aspect, the mass spectrometer is an electrospray ionization mass spectrometer, nano-electrospray ionization mass spectrometer, a triple quadrupole mass spectrometer, a quadrupole mass spectrometer, or an ultra-high mass range hybrid quadrupole mass spectrometer. In a specific aspect, the mass spectrometer comprises a nano-electrospray ionization-mass spectrometer.

These, and other, aspects of the present disclosure will be better appreciated and understood when considered in conjunction with the following description and the accompanying drawings. The following description, while indicating various embodiments and numerous specific details thereof, is given by way of illustration and not of limitation. Many substitutions, modifications, additions, or rearrangements may be made within the scope of the present disclosure.

### BRIEF DESCRIPTION OF THE DRAWINGS

The file of this patent or application contains at least one drawing/photograph executed in color. Copies of this patent or patent application publication with color drawing(s)/photograph(s) will be provided by the Office upon request and payment of the necessary fee.

The summary, as well as the following detailed description, is further understood when read in conjunction with the appended drawings. For the purpose of illustrating the disclosed methods, there are shown in the drawings exemplary embodiments of the methods; however, the methods not limited to the specific embodiments disclosed. In the drawings:

FIG. 1A illustrates the workflow for affinity-resolved SEC-MS, according to an exemplary embodiment.

FIG. 1B illustrates the sample treatment strategy for studying mAb-Fc receptor binding, according to an exemplary embodiment.

FIG. 1C illustrates the sample treatment strategy for studying bsAb-Ag binding, according to an exemplary embodiment.

FIG. 1D illustrates the sample treatment strategy for studying msAb-Ag binding, according to an exemplary embodiment.

FIG. 2 illustrates the workflow for affinity-resolved SEC-MS for a native and variant antibody species, according to an exemplary embodiment.

FIG. 3A shows the SEC-UV traces of FcγRIIIa and mAb1 prepared at ratios from insufficient (1:4) to excess (4:1) levels of FcγRIIIa, according to an exemplary embodiment.

FIG. 3B shows the extracted ion chromatograms for various levels of glycosylation of mAb1 from SEC-PCD-MS analysis of varying ratios, according to an exemplary embodiment. The glycosylation forms include fully glycosylated (FG), partially glycosylated and non-glycosylated.

FIG. 3C shows the deconvoluted mass spectrum of mAb1 from native SEC-MS without FcγRIIIa (top panel), compared to fully glycosylated mAb1 (second panel), partially glycosylated (third panel) and non-glycosylated (bottom panel) from SEC-PCD-MS analysis of the 4:1 mixture sample, according to an exemplary embodiment.

FIG. 4A shows the SEC-UV traces from FcγRIIIa and mAb2 prepared at ratios from insufficient (1:4) to excess (2:1) levels of FcγRIIIa, according to an exemplary embodiment.

FIG. 4B shows the extracted ion chromatograms for various mAb2 glycoforms from SEC-PCD-MS analysis of each mixture sample, according to an exemplary embodiment.

FIG. 4C shows the deconvoluted mass spectrum of mAb2 of mAb2 from native SEC-MS without FcγRIIIa (top panel), compared to species eluting at 10.7-11.4 min (middle panel) and 11.4-12.3 min (bottom panel) from SEC-PCD-MS analysis of the 2:1 mixture sample, according to an exemplary embodiment.

FIG. 5A shows native SEC-UV/MS analysis of mAb3 after IdeS digestion comparing the unmodified and CDR N-glycosylated F(ab′)₂ species and the corresponding deconvoluted mass spectra (inset), according to an exemplary embodiment.

FIG. 5B shows SEC-UV traces from affinity-resolved SEC-MS analysis using Ag3 and F(ab′)₂ mixtures prepared at ratios from insufficient (1:2) to excess (4:1) levels of Ag3, according to an exemplary embodiment.

FIG. 5C shows the extracted ion chromatograms of the unmodified F(ab′)₂ species from SEC-PCD-MS comparing the bound and free forms, according to an exemplary embodiment.

FIG. 5D shows the extracted ion chromatograms of the modified F(ab′)₂ species from SEC-PCD-MS comparing the bound and free forms, according to an exemplary embodiment.

FIG. 6A shows native SEC-UV/MS analysis of mAb4 after IdeS digestion, limited reduction and alkylation comparing the elution profiles of Fab and Fc by UV (solid line) and extracted ion chromatograms (dotted and filled lines), in addition to the deconvoluted mass spectra (inset), according to an exemplary embodiment.

FIG. 6B shows SEC-UV traces from affinity-resolved SEC-MS analysis using Ag4 and Fab mixtures prepared at rations from insufficient (1:2) to excess (5:2) levels of Ag4, according to an exemplary embodiment.

FIG. 6C shows the extracted ion chromatograms of the light chain from SEC-PCD-MS comparing the bound and free forms, according to an exemplary embodiment.

FIG. 6D shows the extracted ion chromatograms of the unmodified Fd from SEC-PCD-MS comparing the bound and free forms, according to an exemplary embodiment.

FIG. 6E shows the extracted ion chromatograms of oxidized Fd from SEC-PCD-MS showing the free form, according to an exemplary embodiment.

FIGS. 7A and 7B show the deconvoluted mass spectra of Fd in complex form with Ag4 (FIG. 7A) or free form (FIG. 7B) at varying ratios from insufficient (1:2) to excess (5:2) levels of Ag4, according to an exemplary embodiment.

FIG. 8 illustrates the effect on the binding affinity of Fc mutations to the FcRn receptor in varying amounts of FcRn receptor, according to an exemplary embodiment.

FIG. 9 shows the SEC-TIC traces from native SEC-MS analysis using the parent mAb-A (top) and two mutations, mAb-A^{YTE} (middle), mAb-A^{LS} (bottom), according to an exemplary embodiment.

FIG. 10 shows the SEC-UV traces from affinity-resolved SEC-MS analysis (performed at pH 5.9) using FcRn and mixtures of mAb-A, mAb-A^{YTE}, and mAb-A^{LS} prepared at different ratios from insufficient (1:6) to excess (6:3) levels of FcRn, according to an exemplary embodiment.

FIG. 11 shows the extracted ion chromatograms of mAb-A, mAb-A^{YTE}, and mAb-A^{LS}, from SEC-PCD-MS comparing the bound and free forms from mixtures prepared at ratios from insufficient (1:6) to excess (6:3) levels of FcRn, with SEC performed at pH 5.9, according to an exemplary embodiment.

FIG. 12 shows the SEC-UV traces from affinity-resolved SEC-MS analysis using FcRn and mAb-A mixtures prepared at different ratios at elevated pH (pH 7.3), according to an exemplary embodiment.

FIG. 13 shows the extracted ion chromatograms of mAb-A, mAb-A^{YTE}, and mAb-A^{LS}, from SEC-PCD-MS comparing the bound and free forms from mixtures prepared at ratios from insufficient (1:6) to excess (6:3) levels of FcRn, with SEC performed at pH 5.9, according to an exemplary embodiment.

FIG. 14A shows the AR-SEC-UV traces of mAb-B wild type, mAb-B with YTE mutations, and mAb-B with LS mutations, prepared at FcRn: mAb mixture ratios from insufficient (1:6, 1:3, 2:3) to excess (4:3, 6:3) levels of FcRn, according to an exemplary embodiment.

FIG. 14B shows the extracted ion chromatograms for various levels of binding from AR-SEC-XIC analysis of varying ratios, according to an exemplary embodiment.

FIG. 14C shows the deconvoluted mass spectrum of mAb-B (top panel), compared to mAb-B with YTE mutations (second panel), and mAb-B with LS mutations (third panel), according to an exemplary embodiment.

FIG. 15 shows calculated depletion rates of each mAb species for wild type, YTE mutant, and LS mutant.

FIG. 16A shows the AR-SEC-UV traces of mAb control and FcRn: mAb-D mixture ratios (1:4, 2:4, 4:4, 6:4, 8:4), according to an exemplary embodiment.

FIG. 16B shows the extracted ion chromatograms for various levels of binding from AR-SEC-XIC analysis of varying ratios, according to an exemplary embodiment.

FIG. 16C shows the deconvoluted mass spectrum of mAb-D control (top panel), compared to mAb-D wild type (second panel), mAb-D with 1×O (third panel), and mAb-D with 2×O (fourth panel), according to an exemplary embodiment.

FIG. 17A is a diagram of an antibody comprising CDR N-glycosylation, according to an exemplary embodiment.

FIG. 17B is a diagram of IdeS digestion, according to an exemplary embodiment.

FIG. 18A shows SEC-UV/MS analysis of mAb3 after IdeS digestion comparing the unmodified and N-glycosylated F(ab′)₂ species and the corresponding deconvoluted mass spectra (inset), according to an exemplary embodiment.

FIG. 18B shows AR-SEC-UV traces from affinity-resolved SEC-MS analysis using Ag3 and F(ab′)₂ mixtures prepared at ratios 1:2, 2:3, 1:1, 2:1, and 3:1 of Ag3:mAb3, according to an exemplary embodiment.

FIG. 18C shows the extracted ion chromatograms of the unmodified F(ab′)₂ species from SEC-PCD-MS comparing the complexed and unbound forms, according to an exemplary embodiment.

FIG. 18D shows the extracted ion chromatograms of the modified F(ab′)₂+G2FS2 species from SEC-PCD-MS comparing the complexed and unbound forms, according to an exemplary embodiment.

FIG. 19A is a diagram of an antibody comprising CDR cysteinylation, according to an exemplary embodiment.

FIG. 19B shows native SEC-UV/MS analysis of Ides-treated mAb5 sample. The chromatography displays the SEC-UV trace. The inset displays the deconvoluted mass spectrum of F(ab′)₂ peak. Prior to SEC-UV/MS analysis, mAb5 was subjected to site-specific digestion with IdeS (1 IUB milliunit per 1 μg of protein) in 50 mM Tris-HCl (pH 7.5) at 37° C. for 1 hour, to generate the F(ab′)₂ and Fc fragments. SEC-UV/MS analysis was performed using an Acquity BEH200 SEC column (4.6×300 mm, 1.7 μm, 200 Å), according to an exemplary embodiment.

FIG. 20A shows SEC-UV traces from Affinity-resolved SEC-MS analysis using Ag5 and F(ab′)₂ mixtures prepared at ratios from insufficient level (1:4) to excess level (4:1) of Ag5.

FIG. 20B shows XICs of the unmodified F(ab′)₂ species and F(ab′)₂ variants with 0-2 cysteinylation and 0-2 trisulfide modification(s) from SEC-PCD-MS analyses of each mixture sample showing their distributions in the complex and the unbound forms. The XICs were reconstructed using the most abundant charge state of each species.

FIG. 20C is a diagram of F(ab′)₂ variants detected by intact MS, according to an exemplary embodiment.

FIG. 21A is a shows SEC-UV/MS analysis of mAb4 after IdeS digestion and DTT/IAA treatment and the corresponding deconvoluted mass spectra (inset), according to an exemplary embodiment.

FIG. 21B shows AR-SEC-UV traces from affinity-resolved SEC-MS analysis using Ag4 and Fab mixtures prepared at ratios 1:2, 1:1, 3:2, 2:1, and 5:2 of Ag4:Fab, according to an exemplary embodiment.

FIG. 21C shows the extracted ion chromatograms comparing the complexed and unbound forms, according to an exemplary embodiment.

FIG. 21D shows the extracted ion chromatograms comparing the complexed and unbound forms, according to an exemplary embodiment.

FIG. 21E shows the extracted ion chromatograms comparing the complexed and unbound forms, according to an exemplary embodiment.

FIG. 21F is a diagram of an antibody comprising CDR Met oxidation, according to an exemplary embodiment.

FIG. 21G is a diagram of IdeS digestion followed by limited reduction (DTT) and alkylation (IAA), according to an exemplary embodiment.

FIG. 22A shows native SEC-UV/MS (panel 1) and native SEC-PCD-UV/MS (panel 2-5) analyses of thermal stressed mAb6 sample. Panels 1 and 2: SEC-UV traces; panel 3: XIC of C-term fragment of HC CDR3 clipped mAb6; panel 4: XIC of N-term fragment of HC CDR3 clipped mAb6; panel 5: XIC of unclipped mAb6.

FIG. 22B shows a deconvoluted mass spectrum of the main peak from SEC-MS analysis of thermal stressed mAb6 sample.

FIG. 22C shows a deconvoluted mass spectrum of the main peak from SEC-PCD-MS analysis of thermal stressed mAb6 sample.

FIG. 22D is a diagram of an antibody comprising CDR clipping, according to an exemplary embodiment.

FIG. 23A is a diagram of IdeS digestion followed by limited reduction (DTT) and alkylation (IAA), according to an exemplary embodiment. The diagram is an experimental scheme of AR-SEC-MS analysis of mAb6 at Fab level, according to an exemplary embodiment.

FIG. 23B shows SEC-UV traces from AR-SEC-MS analysis using Ag6 and Fab mixtures prepared at ratios from insufficient level (Ag6:mAb6=1:5) to excess level (Ag6:mAb6=5:1) of Ag6.

FIG. 23C shows XICs of the LC (red), Fd (blue), Fd N-term fragment (magenta), and Fd C-term fragment (green) from SEC-PCD-MS analyses of each mixture sample showing their distributions in the complex and the unbound forms. The XICs were reconstructed using the most abundant charge state of each species.

FIG. 24 shows SEC-PCD-MS analysis of mAb1 (Ides digested and DTT, IAA treated) and Ag1 mixed at Ag-to-Fab ratio of 1:2 showing SEC-UV trace (black solid line), XIC of Fd (blue filled dotted line), XIC of LC (red filled dotted line), and deconvoluted mass spectra of the complex peak and unbound Fab peak detected with PCD showing mass detection of LC and Fd.

FIG. 25A shows 2D SEC-SCX-MS analysis of protein mixture of mAb1 (IdeS, DTT, IAA treated) and Ag1 at Ag-to-Fab ratio of 1:2. Separation of Ag-Fab mixture by SEC, demonstrated by SEC-UV trace.

FIG. 25B shows flow paths of 2D SEC-SCX-MS at different stages of the operation as controlled by three 6-port valves and two binary pumps.

FIG. 25C shows TIC trace of the 2D SEC-SCX-MS analysis of the Ag-Fab mixture. Valve positions and mobile phase conditions for each separation and analytical phase are marked.

FIG. 26A shows UV traces from 2D AR-SEC-SCX-MS analysis using Ag1 and Fab mixtures prepared at ratios from insufficient level (˜1:4) to excess level (˜1:1) of Ag1.

FIG. 26B shows XICs of LC from 2D SEC-SCX-MS (PCD enabled) analyses of each mixture sample showing its distribution in the complex (5-6 min) and bound forms (17.5-19 min), as well as relative abundances of the CDR deamidated Fab species to the unmodified Fab species in the unbound population.

FIG. 27A shows native SCX-UV/MS analysis of untreated intact trastuzumab sample; UV trace is displayed with assignments of major charge variant peaks.

FIG. 27B shows native SCX-UV/MS analysis of Ides-digested and DTT/IAA treated (inter-chain disulfide bond reduced and alkylated) trastuzumab sample; UV trace is displayed with assignments of major charge variant peaks of the Fab.

FIG. 27C shows deconvoluted mass spectra of charge variant peaks from FIG. 27B acquired both under native conditions (w/o PCD, left panel) and under denaturing conditions (w/PCD, right panel).

FIG. 28A shows UV traces (solid line) and XICs of LC (dotted and filled line) from AR-SEC-SCX-MS analysis using Her2 and trastuzumab Fab mixtures prepared at various Ag-to-Fab ratios.

FIG. 28B shows XICs of LC from same analyses in FIG. 28A showing the later chromatography segment of SCX-PCD-MS analysis of the unbound fraction.

FIG. 28C shows calculated depletion rates of each Fab species from the unbound to complex from AR-SEC-SCX-MS analysis.

FIG. 29A shows 2D SEC-SCX-MS analyses of neat mAb1 Fab sample (top panel) and Ag1-Fab mixture sample prepared at Ag-to-Fab ratio of 1:2 (bottom panel); XICs of the LC from are displayed.

FIG. 29B shows detailed calculation steps using information from FIG. 29A to allow calculation of K_{D} ratio of variant compared to unmodified mAb (K_{D,variant}/K_{D,unmodified}).

FIG. 29C shows a simplified equation for calculating K_{D,variant}/K_{D,unmodified} using information from AR-SEC-SCX-MS analyses in FIG. 29A.

### DETAILED DESCRIPTION

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the herein disclosure. Where a range of numerical values is stated herein as being less than a stated value, the range is nevertheless bounded on its lower end by a non-zero value. It is not intended that the scope of the methods be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise. The term “about” when used in reference to numerical ranges, cutoffs, or specific values is used to indicate that the recited values may vary by up to as much as 10% from the listed value. Thus, the term “about” is used to encompass variations of ±10% or less, variations of ±5% or less, variations of ±1% or less, variations of ±0.5% or less, or variations of ±0.1% or less from the specified value.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “comprising” is intended to include examples encompassed by the terms “consisting essentially of” and “consisting of”; similarly, the term “consisting essentially of” is intended to include examples encompassed by the term “consisting of.”

For a therapeutic mAb, the binding between the paratope from its complementarity-determining regions (CDRs) and the epitope from its therapeutic target, as well as the interactions between its fragment crystallizable (Fc) region and various Fc receptors, can impact its therapeutic functions. Therefore, modifications within these regions can be impactful and warrant thorough evaluations during drug development. For example, PTMs occurring within the mAb CDRs can often hinder its binding to the target by weakening or blocking the interactions between the epitope and the paratope. However, as not all residues in the CDRs participate in binding, PTMs occurring at non-binding residues may not interfere with the target binding activity. In other cases, PTMs located outside of CDRs may indirectly influence target binding through allosteric effects. Therefore, it is important to assess the impact of individual modifications for CQA identification. While both empirical knowledge and computational modeling approaches provide valuable insights, it is valuable to conduct experimental validation to confirm the impact of each modification on mAb function.

The conventional approach to assessing potential CQAs for their impacts on target and/or Fc receptor binding is a highly intricate process. This process involves the enrichment of the attribute-bearing variant followed by in-vitro binding measurement or cell-based potency testing. The variant enrichment step is important yet challenging in this workflow, as it demands the generation of samples containing individual variants with sufficient purity and quantity to allow unambiguous evaluation of binding affinity. To this end, various native liquid chromatography techniques, such as ion exchange chromatography (IEX), hydrophobic interaction chromatography (HIC), and size exclusion chromatography (SEC), are frequently employed to fractionate the desired attribute-bearing variants, owing to their excellent selectivity towards CDR modifications. This approach, however, can be laborious and may be particularly challenging for low-abundance variants. As a result, application of specific stress conditions is sometimes required to artificially produce variants at elevated levels prior to fractionation, which further increases the complexity and the duration of the enrichment process. Finally, despite extensive fractionation efforts, it may not be possible to enrich certain variants to a necessary purity and quantity, making them unsuitable for this workflow.

An emerging alternative for CQA identification without the need for variant enrichment is by online affinity chromatography coupled with mass spectrometry analysis. In this method, mAb molecules undergo affinity-based separation on a column that is immobilized with its therapeutic target (e.g., an antigen) or various Fc receptors. Often, a pH gradient is employed to sequentially elute mAb variants based on their affinity to the immobilized ligands. Subsequent online mass spectrometry analysis allows direct confirmation of the mAb variants based on their signature mass changes. This approach has been successfully employed to assess critical attributes related to mAb binding with various Fc receptors. For example, the relationship between the mAb Fc N-linked glycosylation and its binding with FcγRIIIa has been extensively studied using online FcγRIIIa-MS. These studies offered valuable insights into how Fc N-linked glycosylation can impact the antibody-dependent cellular cytotoxicity (ADCC). Furthermore, the effects of both unintended Fc modifications (e.g., Met252 oxidation) and deliberate amino acid substitutions (e.g., M252Y/S254T/T256E) on antibody binding to the neonatal Fc receptor (FcRn) have been evaluated by online FcRn-MS, which highlighted their influence on mAb serum half-life.

More recently, an antigen-based affinity chromatography mass spectrometry (AC-MS) approach has also been reported to assess attributes related to target binding. Despite the simplicity, the overall applicability of the AC-MS method is largely limited by the availability of the affinity columns. While it is possible to create custom-made affinity columns using various bonding chemistry, this process often comes with high costs and stability issues related to the reagents. In many cases, protein ligands must be engineered to enhance their stability for use in affinity chromatography applications, and combined with modifications which may occur during the immobilization process, this could lead to inconsistent results when compared to a fully native ligand. This may potentially jeopardize the biological relevance of the derived conclusions.

The use of competitive binding to enrich mAb variants with diminished binding affinity for mass spectrometry (MS) detection has emerged as an attractive alternative to conventional CQA identification methods. By introducing insufficient amounts of antigen into mAb samples, mAb variants with impaired antigen binding ability are enriched in the unbound fraction. Following isolation of the bound and unbound fractions, comparative mass spectrometry analysis can provide facile assessment of the criticality of each attribute in a multiplexed fashion. In particular, SEC is known for its ability to preserve protein complexes that have relatively strong binding affinities, with K_{D} values of 10 nM or lower. Therefore, it has been successfully implemented to separate the bound mAb-antigen complexes from the unbound mAb species based on their apparent differences in hydrodynamic radii. Recently, this SEC-based competitive binding strategy has been coupled online with reversed-phase LC-MS analysis in a 2D-LC format. This approach was successfully applied to evaluate the impact of Fab glycosylation on spike protein binding from four anti-SARS-COV-2 antibodies.

The present application provides an affinity-resolved SEC-MS (AR-SEC-MS) workflow that allows characterization of antigen (Ag) and Fc receptor binding-related CQAs in therapeutic mAbs. In an exemplary embodiment, a series of Ag and mAb (or Fc receptor and mAb) mixtures were prepared by introducing Ag to the corresponding mAb samples from insufficient to excess molar amounts. Upon separating the bound and unbound species in each mixture by SEC, online MS detection was utilized to characterize and compare the distribution of each mAb variant in both its bound and unbound forms. Notably, a denaturing solvent was introduced post SEC separation to liberate the mAb species from the mAb-Ag complex to enable direct and unbiased intact mass monitoring. Finally, using the extracted ion chromatograms, the distributions of mAb variants in bound and unbound forms at varying mixing ratios can be used to assess their relative affinities to the target. The validity of this method was first demonstrated using a well-studied model system focusing on the interactions between Fc gamma receptor III-A (FcγRIIIa) and various Fc N-linked glycoforms. Further, two case studies were discussed in detail to showcase the utility of this new method in assessing the impact of CDR modifications on target binding.

In another exemplary embodiment, the present application provides methods to characterize the effects of glycosylation on mAbs on antigen (Ag) and Fc receptor binding-related CQAs in therapeutic mAbs. In another exemplary embodiment, the present application provides methods to characterize the effects of oxidation on mAbs on antigen (Ag) and Fc receptor binding-related CQAs in therapeutic mAbs.

The present application provides methods for the characterization and identification Fc region variants of an mAb and the binding to an Fc receptor using affinity-resolved SEC-MS (AR-SEC-MS).

This disclosure provides methods to satisfy the aforementioned demands by providing methods to identify and characterize the binding of a therapeutic monoclonal antibody to a binding partner to understand the attribute-and-function relationship. The method of the present disclosure provides rapid CQA evaluation in therapeutic monoclonal antibodies. Exemplary embodiments disclosed herein satisfy the aforementioned demands and the long-felt needs.

Unless described otherwise, all technical and scientific terms used herein have the same meaning as commonly understood by one of ordinary skill in the art to which this disclosure belongs. Although any methods and materials similar or equivalent to those described herein can be used in the practice or testing, particular methods and materials are now described.

The term “a” should be understood to mean “at least one” and the term “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule.

As used herein, the term “therapeutic protein” includes any of proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies.

In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., 28 BIOTECHNOLOGY AND GENETIC ENGINEERING REVIEWS 147-176 (2012), the entirety of which is herein incorporated by reference). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG, IgM, IgA1, IgA2, IgD, or IgE. In certain exemplary embodiments the antibody molecule is a full-length antibody (e.g., an IgG1) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In different embodiments of the present disclosure, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” (bsAbs) includes an antibody capable of selectively binding two or more epitopes. Bispecific antibodies generally comprise two different heavy chains with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fe, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entirety of which is herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein, the term “multispecific antibody” refers to an antibody with binding specificities for at least two different antigens. While such molecules normally will only bind two antigens (e.g., bispecific antibodies, bsAbs), antibodies with additional specificities such as trispecific antibody and KIH Trispecific are also contemplated.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies can be prepared using a wide variety of techniques known in the art including the use of hybridoma, recombinant, and phage display technologies, or a combination thereof.

As used herein, a “protein pharmaceutical product” or “biopharmaceutical product” includes an active ingredient which can be fully or partially biological in nature. In one aspect, the protein pharmaceutical product can comprise a peptide, a protein, a fusion protein, an antibody, an antigen, vaccine, a peptide-drug conjugate, an antibody-drug conjugate, a protein-drug conjugate, cells, tissues, or combinations thereof. In another aspect, the protein pharmaceutical product can comprise a recombinant, engineered, modified, mutated, or truncated version of a peptide, a protein, a fusion protein, an antibody, an antigen, vaccine, a peptide-drug conjugate, an antibody-drug conjugate, a protein-drug conjugate, cells, tissues, or combinations thereof.

The terms “peptide,” “protein” and “polypeptide” refer, interchangeably, to a polymer of amino acids and/or amino acid analogs that are joined by peptide bonds or peptide bond mimetics. The twenty naturally-occurring amino acids and their single-letter and three-letter designations are as follows: Alanine A Ala; Cysteine C Cys; Aspartic Acid D Asp; Glutamic acid E Glu; Phenylalanine F Phe; Glycine G Gly; Histidine H His; Isoleucine I He; Lysine K Lys; Leucine L Leu; Methionine M Met; Asparagine N Asn; Proline P Pro; Glutamine Q Gln; Arginine R Arg; Serine S Ser; Threonine T Thr; Valine V Val; Tryptophan w Trp; and Tyrosine Y Tyr.

The present disclosure provides methods of characterizing the binding of a protein to an antigen, the methods comprising:
incubating the antigen with a sample comprising the protein to form a mixture comprising protein-antigen complex and protein that is not bound to the antigen;
separating the mixture on a liquid chromatography column to separate the protein-antigen complex from the protein that is not bound to the antigen and to form an eluate comprising the protein-antigen complex and the protein that is not bound to the antigen;
contacting the eluate with a denaturation solvent to form a denatured sample;
determining a mass of the protein-antigen complex and a mass of the protein that is not bound to the antigen with a mass spectrometer that is coupled to the liquid chromatography column; and
analyzing the mass of the protein-antigen complex and the mass of the protein that is not bound to the antigen to characterize the binding of the protein to the antigen.

The protein can be digested by pepsin, trypsin, Tryp-N, chymotrypsin, Lys-N, Lys-C, Asp-N, Arg-C, Glu-C, papain, IdeS, and variants or combinations thereof prior to the incubating step.

The protein can be digested with IdeS prior to the incubating step.

The protein can be reduced dithiothreitol (DTT), ß-mercaptoethanol, Ellman's reagent, hydroxylamine hydrochloride, sodium cyanoborohydride, tris(2-carboxyethyl)phosphine hydrochloride (TCEP-HCl), or combinations thereof.

The ratio of antigen to protein in the incubating step can be between 1:10 and 10:1.

The protein can be selected from an antibody, a monoclonal antibody, a bispecific antibody, an antibody fragment, an antibody-derived protein, an antigen-binding protein, an antibody-drug conjugate, or a fusion protein.

The liquid chromatography can comprise reversed phase liquid chromatography, ion exchange chromatography, anion exchange chromatography, weak cation exchange chromatography, strong cation exchange chromatography, size exclusion chromatography, affinity chromatography, hydrophobic interaction chromatography, hydrophilic interaction liquid chromatography (HILIC), mixed-mode chromatography, or a combination thereof.

The mobile phase of the liquid chromatography can comprise ammonium acetate.

The denaturation solvent can comprise acetonitrile (ACN), water, and formic acid (FA).

The denaturation solvent can be added at a 1:1 ratio with the eluent.

The antigen can be protein A, protein G, Fcγ receptor, FcγRIIIa, anti-human Fc antibody, neonatal Fc receptor, Fc epsilon RI, anti-idiotype antibody, or complement component C1q.

The liquid chromatography column eluate can comprise 50 mM-75 mM ammonium acetate.

Following the analysis of the mass of the protein-antigen complex and the mass of the protein that is not bound to the antigen, the method can further comprise:
separating the protein that is not bound to the antigen on a strong cation exchange (SCX) chromatography column to separate modified and unmodified protein and to form an eluate comprising modified protein and unmodified protein;
contacting the eluate comprising the modified protein and the unmodified protein with a denaturation solvent to form a denatured sample;
determining a mass of the modified protein and a mass of the unmodified protein with a mass spectrometer that is coupled to the strong cation exchange chromatography column; and
analyzing the mass of the modified protein and the mass of the unmodified protein to determine if the protein that is not bound to the antigen has a molecular modification.

The strong cation exchange chromatography column separation buffer can comprise 20 mM ammonium acetate.

The molecular modification can comprise asparagine deamidation, aspartic acid isomerization, aspartic acid dehydration, or any combination thereof.

The method can be used to characterize the effect of an Fc region variant on the binding of the protein to the antigen.

The present disclosure provides methods for analyzing the effect of glycosylation on the binding of a protein to an antigen, the methods comprise:
incubating the antigen with a sample comprising the protein to form a mixture comprising protein-antigen complex and protein that is not bound to the antigen;
separating the mixture on a liquid chromatography column to separate the protein-antigen complex from the protein that is not bound to the antigen and to form an eluate comprising the protein-antigen complex and the protein that is not bound to the antigen;
contacting the eluate with a denaturation solvent to form a denatured sample;
determining a mass of the protein-antigen complex and a mass of the protein that is not bound to the antigen with a mass spectrometer that is coupled to the liquid chromatography column; and
analyzing the mass of the protein-antigen complex and the mass of the protein that is not bound to the antigen to analyze the effect of glycosylation on the binding of the protein to the antigen.

The present disclosure provides methods of characterizing the binding of a protein to an antigen, the methods comprise:
incubating the antigen with a sample comprising the protein to form a mixture comprising protein-antigen complex and protein that is not bound to the antigen;
separating the mixture on a liquid chromatography column to separate the protein-antigen complex from the protein that is not bound to the antigen and to form an eluate comprising the protein-antigen complex and the protein that is not bound to the antigen;
contacting the eluate with a denaturation solvent to form a denatured sample;
determining a mass of the protein-antigen complex and a mass of the protein that is not bound to the antigen with a mass spectrometer that is coupled to the liquid chromatography column;
analyzing the mass of the protein-antigen complex and the mass of the protein that is not bound to the antigen to characterize the binding of the protein to the antigen;
separating the protein that is not bound to the antigen on a strong cation exchange (SCX) chromatography column to separate modified protein and unmodified protein and to form an eluate comprising modified and unmodified protein;
contacting the eluate comprising the modified protein and the unmodified protein with a denaturation solvent to form a denatured sample;
determining a mass of the modified protein and a mass of the unmodified protein with a mass spectrometer that is coupled to the strong cation exchange chromatography column; and
analyzing the mass of the modified protein and the mass of the unmodified protein to determine if the protein that is not bound to the antigen has a molecular modification.

The molecular modification can comprise asparagine deamidation, aspartic acid isomerization, aspartic acid dehydration, or any combination thereof.

The ratio of receptor to peptide or protein can be 1:4, 1:2, 1:1, 2:1, or 4:1.

The ratio of antigen to peptide or protein can be 1:2, 2:3, 1:1, 2:1, 3:2, 4:1, or 5:2.

The liquid chromatography can comprise size exclusion chromatography.

A splitter can be used to connect the chromatography column and the mass spectrometer.

The splitter can be used after denaturation to divert a low flow to the mass spectrometer and a high flow to a UV detector.

The mass spectrometer can be an electrospray ionization mass spectrometer, nano-electrospray ionization mass spectrometer, a triple quadrupole mass spectrometer, a quadrupole mass spectrometer, or an ultra-high mass range hybrid quadrupole mass spectrometer.

The mass spectrometer can comprise a nano-electrospray ionization-mass spectrometer.

Without wishing to be bound by theory, fully glycosylated antibodies or fragments thereof form complexes with antigen faster than partially glycosylated antibodies and fragments thereof, and the complexes comprising fully glycosylated antibodies or fragments thereof are the most stable, as evidenced by earliest retention time when separated on a size exclusion chromatography column. Partially glycosylated antibodies or fragments thereof form complexes more slowly than fully glycosylated antibodies or fragments thereof and the complexes may exhibit dissociation when separated on an SEC column. Non-glycosylated antibodies or fragments thereof do not readily for antigen complexes, even in the presence of excess antigen.

In contrast, N-glycosylation in the CDR region of an antibody or fragment thereof negatively impacts complexation with antigen.

YTE and LS mutations in the Fc region of an antibody enhance antigen affinity as compared to an antibody with a wild type Fc region.

Without wishing to be bound by theory, deamidation on an antibody CDR decreases binding affinity between the antigen and the antibody. The relative binding affinities of the different CDR modified variant species and unmodified species of antibody are: LC N30 deamidation <HC D102 isomerization <HC D102 cyclization <LC N55 deamidation unmodified.

In some exemplary embodiments, the sample can be prepared prior to LC-MS analysis. Preparation steps can include reduction, denaturation, alkylation, dilution and/or digestion.

Post-translational modification can enhance or reduce complex formation depending on the location of the modification on the peptide or protein as disclosed in the examples herein.

In some embodiments, online denaturation coupled with mass spectrometry can enhance the analysis of the protein-antigen complex and the protein that is not bound to the antigen to characterize the binding of the protein to the antigen and complex formation.

In an exemplary embodiment, the mAb working solutions were prepared at equimolar concentrations. In a specific aspect, the equimolar concentrations of mAbs can be about 30 μM, about 35 μM, about 40 μM, about 45 μM, or about 50 μM. In another exemplary embodiment, the molar ratios of binding partner to antibody are between about 1:10 and 10:1, including any and all combinations in between. In a specific embodiment, the molar ratio of binding partner to antibody is about 1:6, about 1:3, about 1:2, about 1:1, about 2:1, about 2:3, about 3:2, about 4:1, about 4:3, about 5:2, and about 6:3.

In an exemplary embodiment, the pH of the mAb mixture of mAb and binding partner is about pH 5.0, about pH 5.1, about pH 5.2, about pH 5.3, about pH 5.4, about pH 5.5, about pH 5.6, about pH 5.7, about pH 5.8, about pH 5.9, about pH 6.0, about pH 6.1, about pH 6.2, about pH 6.3, about pH 6.4, about pH 6.5, about pH 6.6, about pH 6.7 or about pH 6.8, including any and all values in between. In a specific aspect, the binding of mAb and receptor and/or antigen was about pH 6.8. In another specific aspect, the binding of mAb and FcRn was about pH 5.9.

In an exemplary embodiment, the gel filtration column is equilibrated with ammonium acetate. In a specific aspect, the concentration of ammonium acetate can be between 75 mM ammonium acetate and 300 mM ammonium acetate. In a specific aspect, the concentration of ammonium acetate can be about 75 mM ammonium acetate, about 100 mM ammonium acetate, about 125 mM ammonium acetate, about 130 mM ammonium acetate, about 140 mM ammonium acetate, about 145 mM ammonium acetate, about 150 mM ammonium acetate, about 155 mM ammonium acetate, about 160 mM ammonium acetate, about 165 ammonium acetate, ab out 170 mM ammonium acetate, about 175 mM ammonium acetate, about 200 mM ammonium acetate, about 250 mM ammonium acetate, or about 300 mM ammonium acetate, including any and all values in between. In another specific aspect, the gel filtration is performed at a pH of about pH 5.0, about pH 5.1, about pH 5.2, about pH 5.3, about pH 5.4, about pH 5.5, about pH 5.6, about pH 5.7, about pH 5.8, about pH 5.9, about pH 6.0, about pH 6.1, about pH 6.2, about pH 6.3, about pH 6.4, about pH 6.5, about 6.6, about 6.7 about 6.8, about 6.9 or about 7.0, including any and all values in between. In a specific aspect, a pH of 6.8 was used for gel filtration.

As used herein, the term “affinity” or “affinity-binding molecule” refers to affinity intermolecular interactions, such as the strength of the interaction between a single biomolecule and its binding partner, or ligand. The intermolecular interactions can include non-covalent intermolecular interactions such as hydrogen bonding, electrostatic interactions, hydrophobic and Van der Waals forces between two molecules. Shape complementarity is also crucial for the affinity intermolecular interactions. The possible affinity toward a target molecule can be obtained with a ligand having a mirror image of the shape of the target surface with a complementing charge distribution. Binding affinity, for example, strength of the interactions, can be measured by the equilibrium dissociation constant (Kd) to rank order strengths of bimolecular interactions. The affinity binding of two molecules can be viewed as the strength of the interaction for binding reversibly. The dissociation constant defines the likelihood that an interaction between two molecules will break. (Eaton et al., Let's get specific: the relationship between specificity and affinity, Chemistry & Biology, October 1995, volume 2, No. 10, pages 633-638, Current Biology Ltd, ISSN 1074-5521; Panagiotis et al., 2013, On the binding affinity of macromolecular interactions: daring to ask why proteins interact, Journal of the Royal Society Interface, 10:20120835, http://dx.doi.org/10.1098/rsif.2012.0835). The affinity-binding molecule can be immobilized on a solid surface or a solid phase. By “solid surface” or “solid phase” is meant a non-aqueous matrix to which the affinity-binding molecule can adhere. The solid phase of interest herein can comprise a glass or silica surface. The solid phase may be a purification column or a discontinuous phase of discrete particles.

As used herein, “protein denaturing” or “denaturation” can refer to a process in which the three-dimensional shape of a molecule is changed from its native state. Protein denaturation can be carried out using a protein denaturing agent. Non-limiting examples of a protein denaturing agent include heat, high or low pH, reducing agents like TCEP (see below), or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples of chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

As used herein, the term “protein reducing agent” refers to the agent used for reduction of disulfide bridges in a protein. Non-limiting examples of protein reducing agents used to reduce a protein are dithiothreitol (DTT), ß-mercaptoethanol, Ellman's reagent, hydroxylamine hydrochloride, sodium cyanoborohydride, tris(2-carboxyethyl)phosphine hydrochloride (TCEP-HCl), or combinations thereof. A conventional method of protein analysis, reduced peptide mapping, involves protein reduction prior to LC-MS analysis. In contrast, non-reduced peptide mapping omits the sample preparation step of reduction in order to preserve endogenous disulfide bonds. In some exemplary embodiments, non-reduced preparation may be used, for example, in order to preserve an endogenous disulfide bond between Fab arms of an antibody or antibody-derived protein. In other exemplary embodiments, partially-reduced preparation may be used, for example, in order to reduce the disulfide bond between Fab arms of an antibody or antibody-derived protein without fully reducing the protein.

As used herein, the term “digestion” refers to hydrolysis of one or more peptide bonds of a protein. The methods of present disclosure can include contacting a quenched sample with a hydrolyzing agent. There are several approaches to carrying out digestion of a protein in a sample using an appropriate hydrolyzing agent, for example, enzymatic digestion or non-enzymatic digestion. Digestion of a protein into constituent peptides can produce a “peptide digest” that can further be analyzed using peptide mapping analysis.

As used herein, the term “digestive enzyme” or “hydrolyzing agent” refers to any of a large number of different agents that can perform digestion (enzymatically and non-enzymatically) of a protein. Non-limiting examples of hydrolyzing agents that can carry out enzymatic digestion include protease from Aspergillus saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N (Asp-N), endoproteinase Arg-C (Arg-C), endoproteinase Glu-C (Glu-C) or outer membrane protein T (OmpT), immunoglobulin-degrading enzyme of Streptococcus pyogenes (IdeS), IdeZ, IgdE, glyserias, thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, 12 JOURNAL OF PROTEOME RESEARCH 1067-1077 (2013)). One or a combination of hydrolyzing agents can cleave peptide bonds in a protein or polypeptide, in a sequence-specific manner, generating a predictable collection of shorter peptides. The ratio of hydrolyzing agent to protein and the time required for digestion can be appropriately selected to obtain optimal digestion of the protein. When the enzyme to substrate ratio (E/S) is unsuitably high, the correspondingly high digestion rate will not allow sufficient time for the peptides to be analyzed by mass spectrometer, and sequence coverage will be compromised. On the other hand, a low E/S ratio would need long digestion and thus long data acquisition time. The enzyme to substrate ratio can range from about 1:0.5 to about 1:200. As used herein, the term “digestion” refers to hydrolysis of one or more peptide bonds of a protein.

As used herein, the term “liquid chromatography” refers to a process in which a biological and/or chemical mixture carried by a liquid can be separated into components as a result of differential distribution of the components as they flow through (or into) a stationary liquid or solid phase. Non-limiting examples of liquid chromatography include reverse phase liquid chromatography, ion-exchange chromatography, size exclusion chromatography, affinity chromatography, hydrophobic interaction chromatography, hydrophilic interaction chromatography, or mixed-mode chromatography. In some aspects, the sample or eluate can be subjected to any one of the aforementioned chromatographic methods or a combination thereof.

In some exemplary embodiments, the chromatography can be size-exclusion chromatography.

As used herein, the terms “SEC chromatography resin” or “SEC chromatography media” are used interchangeably and can include any kind of solid phase used in SEC which separates the impurity from the desired product (e.g., a homodimer contaminant for a bispecific antibody product). The volume of the resin, the length and diameter of the column to be used, as well as the dynamic capacity and flow-rate can depend on several parameters such as the volume of fluid to be treated, concentration of protein in the fluid to be subjected to the process.

In an exemplary embodiment, the post-column denaturation was carried out using a denaturation solvent comprised of acetonitrile (ACN), water and formic acid (FA). In a specific aspect, the solvent consists of about 40% ACN, about 50% ACN, about 60% ACN, about 70% ACN, or about 80% ACN, including any and all values in between. In a specific aspect, the solvent consists of about 0% FA, about 1% FA, about 2% FA, about 3% FA, about 4% FA, about 5% FA, about 6% FA, about 7% FA or about 8% FA. In a further specific aspect, water is used to bring the final percentage of solvents up to 100%.

As used herein, the term “mass spectrometer” includes a device capable of identifying specific molecular species and measuring their accurate masses. The term is meant to include any molecular detector into which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application.

The mass spectrometer can be coupled to a liquid chromatography-multiple reaction monitoring system. More generally, a mass spectrometer may be capable of analysis by selected reaction monitoring (SRM), including consecutive reaction monitoring (CRM) and parallel reaction monitoring (PRM).

As used herein, “multiple reaction monitoring” or “MRM” refers to a mass spectrometry-based technique that can precisely quantify small molecules, peptides, and proteins within complex matrices with high sensitivity, specificity and a wide dynamic range (Paola Picotti & Ruedi Aebersold, Selected reaction monitoring-based proteomics: workflows, potential, pitfalls and future directions, 9 NATURE METHODS 555-566 (2012)). MRM can be typically performed with triple quadrupole mass spectrometers wherein a precursor ion corresponding to the selected small molecules/peptides is selected in the first quadrupole and a fragment ion of the precursor ion was selected for monitoring in the third quadrupole (Yong Seok Choi et al., Targeted human cerebrospinal fluid proteomics for the validation of multiple Alzheimers disease biomarker candidates, 930 JOURNAL OF CHROMATOGRAPHY B 129-135 (2013)).

SRM/MRM/Selected-ion monitoring (SIM) is a method used in tandem mass spectrometry in which an ion of a particular mass is selected in the first stage of a tandem mass spectrometer and an ion product of a fragmentation reaction of the precursor ion is selected in the second mass spectrometer stage for detection. Examples of triple quadrupole mass spectrometers (TQMS) that can perform MRM/SRM/SIM include but are not limited to QTRAP® 6500 System (Sciex), QTRAP® 5500 System (Sciex), Triple QTriple Quad 6500 System (Sciex), Agilent 6400 Series Triple Quadrupole LC/MS systems, and Thermo Scientific™ TSQ™ Triple Quadrupole system.

In addition to MRM, the choice of peptides can also be quantified through Parallel-Reaction Monitoring (PRM). PRM is the application of SRM with parallel detection of all transitions in a single analysis using a high-resolution mass spectrometer. PRM provides high selectivity, high sensitivity and high-throughput to quantify selected peptides (Q1), and hence quantify proteins. Multiple peptides can be specifically selected for each protein. PRM methodology can use the quadrupole of a mass spectrometer to isolate a target precursor ion, fragment the targeted precursor ion in the collision cell, and then detect the resulting product ions in the Orbitrap mass analyzer. PRM can use a quadrupole time-of-flight (QTOF) or hybrid quadrupole-orbitrap (QOrbitrap) mass spectrometer to carry out the identification of peptides and/or proteins. Examples of QTOF include but are not limited to TripleTOF® 6600 System (Sciex), TripleTOF® 5600 System (Sciex), X500R QTOF System (Sciex), 6500 Series Accurate-Mass Quadrupole Time-of-Flight (Q-TOF) (Agilent) and Xevo G2-XS QT of Quadrupole Time-of-Flight Mass Spectrometry (Waters). Examples of QObitrap include but are not limited to Q Exactive™ Hybrid Quadrupole-Orbitrap Mass Spectrometer (Thermo Scientific) and Orbitrap Fusion™ Tribrid™ (Thermo Scientific).

Non-limiting advantages of PRM include: elimination of most interferences; providing more accuracy and attomole-level limits of detection and quantification; enabling the confident confirmation of the peptide identity with spectral library matching; reducing assay development time since no target transitions need to be preselected; and ensuring UHPLC-compatible data acquisition speeds with spectrum multiplexing and advanced signal processing.

The mass spectrometer in the methods or systems of the present application can be, for example, an electrospray ionization mass spectrometer, nano-electrospray ionization mass spectrometer, or a triple quadrupole mass spectrometer, wherein the mass spectrometer can be coupled to a liquid chromatography system, wherein the mass spectrometer is capable of performing LC-MS (liquid chromatography-mass spectrometry) or LC-PRM-MS (liquid chromatography-parallel reaction monitoring-mass spectrometry) analyses.

In some exemplary embodiments, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. MS/MS, or MS2, can be performed by first selecting and isolating a precursor ion (MS1), and fragmenting it to obtain meaningful information. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device.

The peptides identified by the mass spectrometer can be used as surrogate representatives of the intact protein and their post-translational modifications. They can be used for protein characterization by correlating experimental and theoretical MS/MS data, the latter generated from possible peptides in a protein sequence database. The characterization includes, but is not limited to, identifying the protein, sequencing amino acids of the protein fragments, determining protein sequencing, quantifying the protein, locating post-translational modifications, identifying post translational modifications, or comparability analysis, or combinations thereof.

As used herein, the term “electrospray ionization” or “ESI” refers to the process of spray ionization in which either cations or anions in solution are transferred to the gas phase via formation and desolvation at atmospheric pressure of a stream of highly charged droplets that result from applying a potential difference between the tip of the electrospray needle containing the solution and a counter electrode. There are generally three major steps in the production of gas-phase ions from electrolyte ions in solution. These are: (a) production of charged droplets at the ES infusion tip; (b) shrinkage of charged droplets by solvent evaporation and repeated droplet disintegrations leading to small highly charged droplets capable of producing gas-phase ions; and (c) the mechanism by which gas-phase ions are produced from very small and highly charged droplets. Stages (a)-(c) generally occur in the atmospheric pressure region of the apparatus.

In some exemplary aspects, the mass spectrometer can use nanoelectrospray or nanospray ionization. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools”. Bioinformatics tools provide the capacity to search uninterpreted MS/MS spectra against all possible sequences in the database(s), and provide interpreted (annotated) MS/MS spectra as an output. Non-limiting examples of such tools are Mascot (matrixscience.com), Spectrum Mill (chem.agilent.com), PLGS (waters.com), PEAKS (bioinformaticssolutions.com), Proteinpilot (download.appliedbiosystems.com/proteinpilot), Phenyx (phenyx-ms.com), Sorcerer (sagenresearch.com), OMSSA (pubchem.ncbi.nlm.nih.gov/omssa/), X!Tandem (thegpm.org/TANDEM/), Protein Prospector (prospector.ucsf.edu/prospector/mshome.htm), Byonic (proteinmetrics.com/products/byonic) or Sequest (fields.scripps.edu/sequest).

An alternative embodiment of the present disclosure includes methods of identifying or characterizing the effect of glycosylation on the binding of a peptide or protein to a binding partner, comprising contacting a sample comprising the peptide or protein with a binding partner in varying ratios to form a complex, contacting the complex to a liquid chromatography column to separate bound and free material in an eluate, contacting the eluate to a denaturant to form a denatured sample, contacting the denatured sample to a mass spectrometer that is coupled to the liquid chromatography column, to determine the mass of the bound and free material; and analyzing the mass of the bound and free material to identifying the binding of a peptide or protein to a binding partner.

An alternative embodiment of the present disclosure includes methods of identifying or characterizing the effect of an Fc region variant on the binding of a peptide or protein to a binding partner, comprising contacting a sample comprising the peptide or protein with a binding partner in varying ratios to form a complex, contacting the complex to a liquid chromatography column to separate bound and free material in an eluate, contacting the eluate to a denaturant to form a denatured sample, contacting the denatured sample to a mass spectrometer that is coupled to the liquid chromatography column, to determine the mass of the bound and free material; and analyzing the mass of the bound and free material to identifying the binding of a peptide or protein to a binding partner.

It is understood that the present disclosure is not limited to any of the aforesaid protein(s), therapeutic protein(s), antibody(s), recombinant protein(s), protein pharmaceutical product(s), sample(s), protein alkylating agent(s), protein denaturing agent(s), protein reducing agent(s), digestive enzyme(s), chromatographic method(s), mass spectrometer(s), database(s), bioinformatics tool(s), pH range(s) or value(s), temperature(s), or concentration(s), and any protein(s), therapeutic protein(s), antibody(s), recombinant protein(s), protein pharmaceutical product(s), sample(s), protein alkylating agent(s), protein denaturing agent(s), protein reducing agent(s), digestive enzyme(s), chromatographic method(s), mass spectrometer(s), database(s), bioinformatics tool(s), pH, temperature(s), or concentration(s) can be selected by any suitable means.

The present disclosure will be more fully understood by reference to the following examples. They should not, however, be construed as limiting the scope of the disclosure.

### EXAMPLES

### Materials and Methods

Materials. Deionized water was provided by a Milli-Q integral water purification system installed with a MilliPak Express 20 filter (Millipore Sigma, Burlington, MA). Ammonium acetate (LC/MS grade) and 2-propanol (IPA; HPLC grade) was purchased from Sigma-Aldrich (St. Louis, MO). FabRICATOR® was purchased from Genovis (Cambridge, MA). Invitrogen UltraPure 1 M Tris-HCl buffer, pH 7.5, Pierce™ DTT (Dithiothreitol, No-Weigh™ Format), iodoacetamide (IAA), and Acetonitrile (ACN; Optima LC/MS grade) were obtained from Thermo Fisher Scientific (Waltham, MA). Formic acid (FA, 98-100%, Suprapur for trace metal analysis) was purchased from Millipore Sigma (Burlington, MA). Recombinant human FcγRIIIa, mAb1, mAb2, mAb3, mAb4, Ag3 (target of mAb3) and Ag4 (target of mAb4) were all produced at Regeneron (Tarrytown, NY).

Sample Preparation. To prepare mixtures of FcγRIIIa and mAb samples, FcγRIIIa, mAb1 and mAb2 working solutions were first prepared at equal molar concentrations of 40 μM, followed by mixing the corresponding working solutions at FcγRIIIa-to-Ab molar ratios of 1:4, 1:2, 1:1, 2:1, and 4:1, respectively. To prepare the antigen and antibody mixtures, mAb3 and mAb4 samples were first subjected to site-specific digestion with FabRICATOR (1 IUB milliunit per 1 μg of protein) in 50 mM Tris-HCl (pH 7.5) at 37° C. for 1 hour, to generate the F(ab)′₂ and Fc fragments. The digestion products of mAb3 were then mixed with Ag3 at Ag-to-Ab molar ratios of 1:2, 2:3, 1:1, 2:1, and 4:1, respectively. The digestion products of mAb4 were further subjected to limited reduction by incubating with 5 mM DTT in 50 mM Tris-HCl (pH 7.5) at 37° C. for 30 min to selectively reduce inter-chain disulfide bonds, followed by alkylation with 20 mM IAA at room temperature in the dark for 30 min. The FabRICATOR digested and partially reduced sample of mAb4 was then mixed with Ag4 at Ag-to-Fab molar ratios of 1:2, 1:1, 3:2, 2:1, and 5:2, respectively. The total protein concentrations for all mixtures were in the range of 1-5 mg/mL.

Affinity-Resolved SEC-MS. The mixtures containing pre-treated mAb and its binding partner (e.g., FcγRIIIa or Ag) at varying molar ratios were subjected to post-column denaturation assisted SEC-MS (SEC-PCD-MS) analysis. Native SEC chromatography was performed on an UltiMate 3000 UHPLC System (Thermo Fisher Scientific, Bremen, Germany) equipped with an Acquity BEH200 SEC column (4.6×150 or 4.6×300 mm, 1.7 μm, 200 Å; Waters, Milford, MA) with the column compartment set to 30° C. An isocratic flow of 150 mM ammonium acetate at 0.2 mL/mL was applied to elute and separate the bound complexes from the unbound mAb or its binding partners in the mixtures. To enable post-column denaturation, a denaturing solvent consisting of 60% ACN, 36% water, and 4% FA was delivered by a secondary pump at a flow rate of 0.2 mL/min and then mixed with the SEC eluent (1:1 mixing) using a T-mixer prior to MS detection. The combined flow (0.4 mL/min) was then split into a microflow (<10 μL/min) for nano-electrospray ionization (NSI)-MS detection and a remaining high flow for UV detection (280 nm). A Thermo Orbitrap Exploris 480 Mass Spectrometer (Thermo Fisher Scientific, Bremen, Germany) equipped with a Microflow-Nanospray Electrospray Ionization (MnESI) Source and a Microfabricated Monolithic Multi-nozzle (M3) emitter (Newomics, Berkeley, CA) was used for MS analysis. A detailed experimental setup and instrument parameters can be found in Yan, Y.; Xing, T.; Wang, S.; Li, N. Versatile, Sensitive, and Robust Native LC-MS Platform for Intact Mass Analysis of Protein Drugs. J Am Soc Mass Spectrom 2020, 31 (10), 2171-2179. DOI: 10.1021/jasms.0c00277 from NLM Medline and Yan, Y.; Xing, T.; Liu, A. P.; Zhang, Z.; Wang, S.; Li, N. Post-Column Denaturation-Assisted Native Size-Exclusion Chromatography-Mass Spectrometry for Rapid and In-Depth Characterization of High Molecular Weight Variants in Therapeutic Monoclonal Antibodies. J Am Soc Mass Spectrom 2021, 32 (12), 2885-2894. DOI: 10.1021/jasms.1c00289 from NLM Medline.

Mixtures of FcRn and mAbs with different Fc regions at varying molar ratios were subjected to post-column denaturation assisted SEC-MS (SEC-PCD-MS) analysis. Native SEC chromatography was performed on a Vanquish Flex UPLC system with a Waters Acquity BEH200 SEC column. A native buffer flow at 0.2 mL/min was applied to separate bound complexes from unbound mAb or FcRn. Post-column denaturation was enabled by a solvent containing 60% acetonitrile and 40% water, delivered at 0.2 mL/min and mixed with the SEC eluent using a T-mixer. The combined flow was then split into a microflow for nano-ESI-MS detection and a high flow for UV detection. A Thermo Orbitrap Exploris 480 mass spectrometer with a NewOmics MnESI ion source was used for MS analysis.

Data analysis. Integration of the XIC peak from the SEC-PCD-MS analysis was performed using Thermo Fisher Xcalibur software (version 3.0). Intact mass spectra from SEC-PCD-MS analysis were deconvoluted using Intact Mass™ software from Protein Metrics.

### Example 1. Affinity-Resolved SEC-MS Workflow

The neonatal Fc receptor (FcRn) and FcγRIIIa are both Fc receptors that play critical roles in the pharmacokinetics (PK) and therapeutic efficacy of monoclonal antibodies (mAbs), respectively. The inventors of the present disclosure devised a method to understand how specific amino acid substitutions, unintended Fc modifications, and N-linked glycosylations, in the Fc region of mAbs can influence their interaction with FcRn and FcγRIIIa, respectively. A novel affinity-resolved size exclusion chromatography-mass spectrometry method for studying attribute-and-function relationship is described. This approach was applied to provide assessment of the impact of different Fc engineering or post translational modifications of antibodies on the Fc receptors affinity in a multiplexed manner.

Assessment of critical quality attributes (CQAs) is an important aspect during the development of therapeutic monoclonal antibodies (mAbs). Attributes that affect either the target binding or Fc receptor engagement may have direct impacts on the drug safety and efficacy, and thus are considered as CQAs.

Affinity-resolved size exclusion chromatography-mass spectrometry (AR-SEC-MS) was developed and this method features wide applicability, fast turn-around, high multiplexity, and approachable implementation.

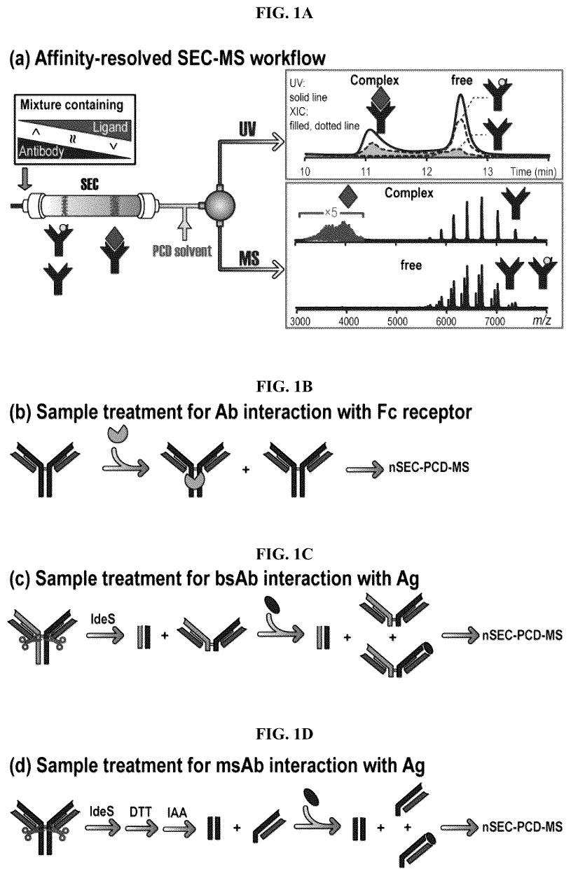

To assess the impact of mAb attributes on target or Fc receptor binding affinity, protein mixtures containing the mAb and its binding partner (e.g., antigen or Fc receptors) were mixed at varying ratios and subjected to SEC-PCD-MS analysis (FIG. 1A). Using a previously reported native LC-MS platform, the bound complex and the unbound mAb species were first separated by SEC based on their differences in hydrodynamic radii. Subsequently, a denaturing solvent was introduced post-column to instantly dissociate the non-covalently bound complex into its constituent mAb and its binding partner prior to MS detection. Next, online MS analysis provided direct identification of mAb proteoforms (e.g., both unmodified and variant forms) based on accurate mass measurement. Finally, using extracted ion chromatogram (XIC), the distribution of each identified mAb proteoform originating from the complex and unbound forms were readily reconstructed and visualized (FIG. 1A). mAb and its binding partner were mixed at varying ratios and subjected to SEC-MS analysis with post-column denaturation (SEC-PCD-MS).

Under competitive binding conditions, that mAb proteoforms with higher binding affinities (e.g., unmodified mAb) will be preferentially enriched into the complex form, while the proteoforms with lower binding affinities (e.g., mAb variants) will be enriched into the unbound form.

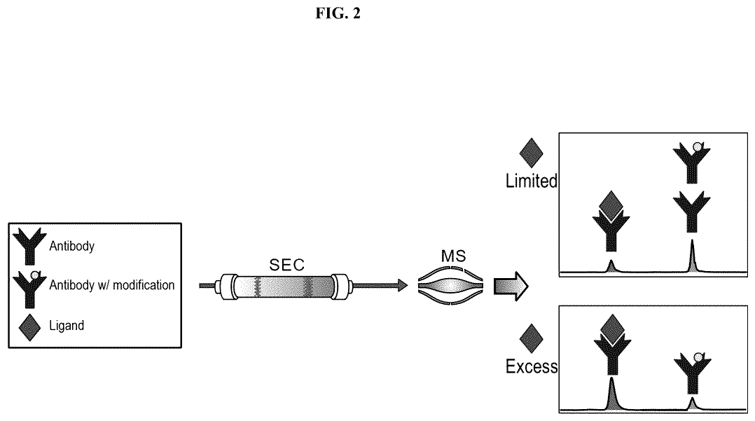

In addition, by limiting conditions of ligand, the unmodified antibody will preferentially bind the antibody, resulting in enrichment of the variant species, as shown in FIG. 2. In addition, in an excess of ligand, the bound species is enriched as compared to the unbound variant species (FIG. 2). By analyzing a series of mixtures at varying mixing ratios, the binding affinities of different mAb variants relative to the unmodified mAb can be simultaneously compared. This workflow can be applied to interrogate the criticality of PTMs in unfractionated and undisturbed samples at various stages of development. With this SEC-PCD-MS workflow, it is only necessary to monitor the mAb species by MS, allowing the often highly heterogeneous antigens or receptors to be excluded from the analysis. In addition, the employed SEC-PCD-MS method produces native-like spectra for mAb molecules, which greatly simplifies the data interpretation, due to the separation of mAb signal from its binding partners in the m/z space (FIG. 1A).

To facilitate data interpretation, three different mAb treatment procedures were performed prior to the mixing with the binding partners. As illustrated in FIG. 1B, when studying the mAb interaction with Fc receptors, the mAb sample was directly mixed with various Fc receptors at different molar ratios without any treatment. In the study of mAb interaction with target antigen, two distinctive sample treatment strategies were adopted for bispecific antibody (bsAb) (FIG. 1C) and monospecific antibody (msAb) (FIG. 1D), respectively. Since the antigen binding only involves the Fab domains of an antibody, both bsAb and msAb molecules were subjected to FabRICATOR (IdeS) treatment to release the F(ab)′₂ fragments prior to the mixing with the target antigen. This treatment significantly improved the MS detection sensitivity by reducing the size of the analytes (from intact mAb to F(ab)′₂ fragment) and removing mass heterogeneities introduced by Fc N-linked glycans. This is particularly crucial for the successful detection of mAb variants that are present in low abundance. For bsAb, each of its two Fab arms can be individually assessed using the corresponding antigen. Due to a simple 1:1 binding stoichiometry between each Fab arm and its target (in most cases), the IdeS-digested sample was directly mixed with the antigen to prepare the mixtures.

In contrast, due to the bivalency, msAb has the potential to form a variety of complexes in the presence of its antigen. These can range from 1:1 and 1:2 complexes to larger aggregates through a process known as “paper-dolling.” This can lead to complicated separation profiles during SEC analysis, posing challenges in interpreting the results. To simplify the binding stoichiometry, the IdeS-digested msAb sample was further subjected to reduction under native conditions to selectively disrupt the inter-chain disulfide bonds and further convert F(ab)′₂ fragments into Fab fragments, which consist of non-covalently interacting Fd and LC. The newly generated free thiols were also capped by an alkylation reaction to prevent disulfide bond reformation. After these treatments, the sample was mixed with the antigen to form a desired 1:1 Fab-Ag complex and subjected to SEC-PCD-MS analysis. Notably, the non-covalent Fab fragments can be well preserved during SEC separation. However, under PCD conditions, they will be further dissociated into Fd and LC for MS detection, due to the absence of inter-chain disulfide bonds. Consequently, further details about the location of the attributes can also be obtained using this sample preparation approach.

### Example 2. Study the Influence of Fe N-Linked Glycosylation on FcγRIIIa Binding by Affinity-Resolved SEC-MS

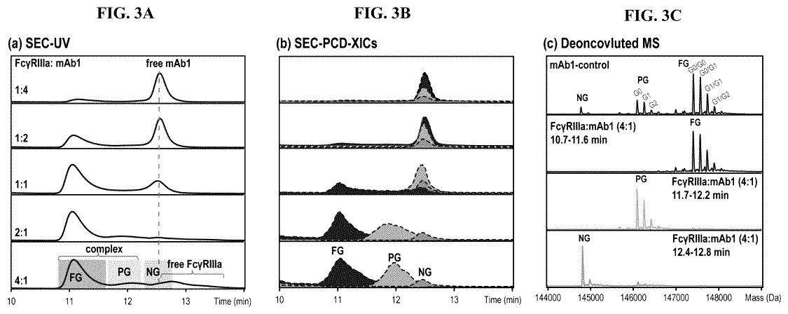

To demonstrate the effectiveness of the affinity-resolved SEC-MS method for studying the attribute-and-function relationship, the interaction between FcγRIIIa and two IgG1 mAbs were subjected to the test. FcγRIIIa, a well-studied Fc receptor with a medium affinity to the Fc domain of IgG molecules (K_{D}˜10-400 nM), is associated with the antibody-dependent cell-mediated cytotoxicity (ADCC) pathway. It binds to the antibody Fc region in a 1:1 stoichiometry and the binding affinity is largely influenced by the composition of the antibody Fc N-glycans at the conserved N-glycosylation site N297 (EU numbering system) from the CH2 domain. In the first study, an IgG1 molecule (mAb1), which was primarily produced as afucosylated antibody, was subjected to affinity-resolved SEC-MS analysis with FcγRIIIa. Briefly, FcγRIIIa and mAb1 were mixed at various ratios (1:4, 1:2, 1:1, 2:1, and 4:1) and the formed mAb-FcγRIIIa complex was separated from the unbound FcγRIIIa or the unbound mAb on an SEC column, followed by online PCD-MS detection (FIG. 3). Upon mixing, a discrete SEC peak (10.7-11.6 min, FIG. 3A) eluting earlier than both the unbound FcγRIIIa and mAb was observed, indicating the formation of a stable mAb-FcγRIIIa complex under SEC conditions. This aligns well with the enhanced binding affinity between FcγRIIIa and an afucosylated IgG1. As the ratio of FcγRIIIa to mAb1 was increased from insufficient levels (1:4 and 1:2) to excess levels (2:1 and 4:1), a corresponding increase in the complex formation and a decrease in unbound mAb abundance was observed. (FIG. 3A).

Using online PCD-MS detection, the distributions of mAb1 glycoforms in the complex and the unbound forms can be readily visualized through XICs. For example, by generating the XICs of the non-glycosylated (NG), partially glycosylated (PG), and fully glycosylated (FG) mAb species in each mixture sample, it was found that the FG species (FIG. 3B, blue trace) displayed the highest binding affinity to the receptor and was transitioned first from the unbound form to the complex form. The PG species (FIG. 3B, cyan trace) only exhibited receptor binding in samples with excess levels of receptors (i.e., 2:1 and 4:1 mixtures), after the FG species were completely transitioned into the complex form. In contrast, the NG species (FIG. 3B, green trace) exhibited no binding to the receptor and remained in the unbound form, even in the presence of excess amounts of FcγRIIIa in the 2:1 and 4:1 mixtures. Despite the similar size, the complex formed by FcγRIIIa and PG species eluted at a significantly later retention time window (12.4-12.8 min) compared to the complex formed by FcγRIIIa and FG species (11.7-12.2 min). This is likely attributed to the weaker binding affinity between the PG species and the FcγRIIIa. As a result, when tested under nonequilibrium conditions, the complex underwent on-column dissociation and the resulting constituents moved at a slower pace than the original complex due to their smaller sizes. This observation also reconciles with a previous report that described dissociation of weakly bound complexes during SEC separation, leading to prolonged retention time. Finally, using the 4:1 mixture sample as an example, the three major glycoforms (NG: 12.4-12.8 min; PG: 11.7-12.2 min; and FG: 10.7-11.6 min) of mAb1 were effectively separated on SEC in an affinity-resolved manner prior to PCD-MS detection (FIG. 3C). The relative affinity of each glycoform to FcγRIIIa binding, as determined in this study, aligned well with previous reports using other techniques. The AR SEC-MS method showed the effect of Fe glycan on FcγRIIIa binding, with relative affinity of each glycoforms to FcγRIIIa being FG (fully glycosylated)>PG (partially glycosylated)>NG (non-glycosylated).

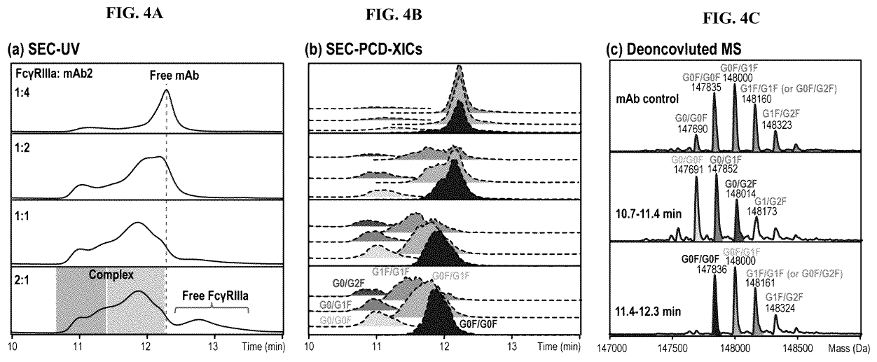

In addition to glycan occupancy (e.g., macroheterogeneity), the affinity of IgG1 antibodies to FcγRIIIa also depends on the specific type of N-glycans (e.g., microheterogeneity) at position N297. In particular, it was previously reported that IgG1 species, which either lack core fucosylation or contain more terminal galactoses on the Fc N-glycan, show increased affinity towards FcγRIIIa. To further demonstrate the ability of the affinity-resolved SEC-MS method in revealing the impact of N-glycan microheterogeneity on antibodies' affinity towards FcγRIIIa, another IgG1 molecule (mAb2) was tested. Unlike mAb1, mAb2 was produced without tampering the fucosylation pathway so that it contains primarily core-fucosylated and biantennary N-glycans (e.g., G0F, G1F, and G2F), with relatively low levels of afucosylated forms (e.g., G0, G1, and G2). Similarly, mixtures of FcγRIIIa and mAb2 prepared at different ratios (1:4, 1:2, 1:1, and 2:1) were separated on SEC and followed by online PCD-MS analysis (FIG. 4). Contrary to the mAb1 example where discrete complex peaks were observed, the complexes formed by mAb2 and FcγRIIIa were eluted within a broad (10.7-12.3 min) and undefined peak. This difference is presumably attributed to the increased on-column dissociation of the mAb2-FcγRIIIa complexes due to the overall reduced affinity of core-fucosylated mAb towards FcγRIIIa. To compare the FcγRIIIa binding affinity, the distribution profiles of mAb glycoforms were reconstructed using the corresponding XICs from the PCD-MS analysis and shown in FIG. 4B. When the amount of FcγRIIIa was very limited (in 1:4 mixture), the singly core-fucosylated glycoforms (G0/GxF, x=0, 1, 2; FIG. 4B, yellow, orange, and red traces) were the first to transition into the complex form, indicating they were preferentially bound to FcγRIIIa. When additional FcγRIIIa was introduced (in 2:1, 1:1, and 2:1 mixtures), the remaining doubly core-fucosylated glycoforms (GxF/GxF, x=0, 1, 2; FIG. 4B, blue, cyan, and green traces) also started to form complexes with FcγRIIIa. In addition, the complexes formed by FcγRIIIa and doubly core-fucosylated glycoforms (GxF/GxF) eluted significantly later (11.4-12.3 min) compared to the complexes formed by FcγRIIIa and singly core-fucosylated glycoforms (G0/GxF; 10.7-11.4 min). This is likely attributed to the increased on-column dissociation between FcγRIIIa and doubly core-fucosylated glycoforms due to reduced binding affinity. The AR SEC-MS method showed that the presence of core fucose reduces Fc binding to FcγRIIIa while end galactose enhances Fc binding to FcγRIIIa (FIG. 4B and FIG. 4C). Singly core-fucosylated species (G0/G0F, G0/G1F, and G0/G2F) formed the complexes the fastest, and the complexes exhibited greater stability during SEC separation (e.g., earliest retention time) (FIG. 4A-4C). Doubly core-fucosylated species (G0F/G0F, G0F/G1F, and G1F/G1F) formed the complexes slower, and the complexes showed increased on-column dissociation during SEC separation (e.g., delayed retention time) (FIG. 4A-4C). Within each group, terminal galactose increases binding affinity to a lesser extent.

By examining the retention order of the three individual glycoforms within each group (e.g., singly core-fucosylated and doubly core-fucosylated), it was also concluded that glycoforms with higher levels of terminal galactose demonstrated higher binding affinities towards FcγRIIIa. This was evidenced by the earlier retention time of their corresponding complexes (or increased stability) during SEC separation compared to those with lower levels of terminal galactose (e.g., elution order: G0/G2F>G0/G1F>G0/G0F; G1F/G1F>G0F/G1F>G0F/G0F). These findings are highly consistent with previous studies highlighting the critical role of Fc N-glycans in influencing FcγRIIIa binding. Finally, using the 2:1 sample as an example, the deconvoluted mass spectra of mAb2 glycoforms eluting from 10.7 to 11.4 min and from 11.4 to 12.3 min were generated to illustrate the affinity-resolved separation of singly and doubly core-fucosylated species (FIG. 4C, middle and bottom panels). Several singly core-fucosylated glycoforms (e.g., G0/G1F, G0/G2F, and G1/G2F) can only be confidently identified by this affinity-resolved SEC-MS method. In contrast, they were not discerned by regular SEC-MS analysis due to their low abundances and similar masses to other more prevalent, doubly core-fucosylated forms (e.g., G0/G1F and G0F/G0F differ by only 16 Da) (FIG. 4C, top panel). Therefore, besides studying the attribute-and-function relationship, this affinity-resolved SEC-MS method can also be a valuable addition to conventional intact mass techniques for mAb heterogeneity characterization, owing to its unique affinity-based separation mechanism.

### Example 3. Evaluation of a CDR N-Linked Glycosylation in a Bispecific Antibody by Affinity-Resolved SEC-MS

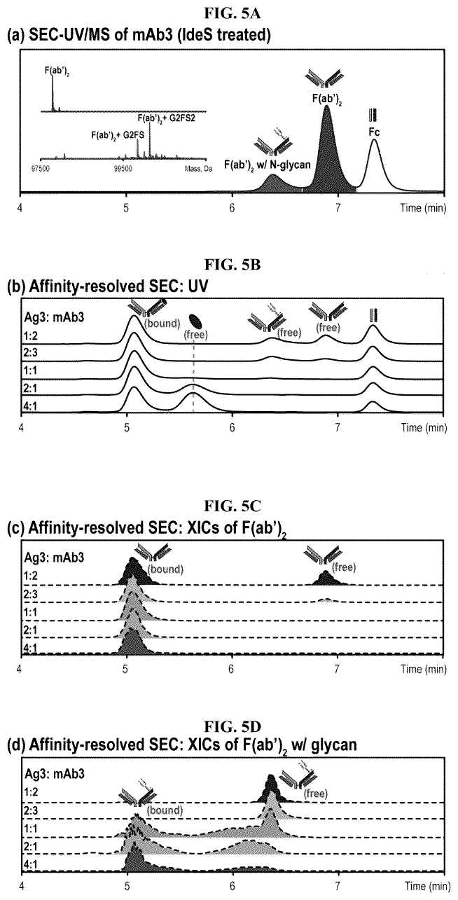

The affinity-resolved SEC-MS method was used to evaluate the impact of a CDR N-linked glycosylation on target binding in a bsAb molecule (mAb3). MAb3 consists of two identical light chains (LC) and two different heavy chains (HC and HC*). This molecule contains a notable level of N-linked glycosylation at the Asn55 residue within the HC CDR2, which is known to reduce the binding affinity to its corresponding target (referred to as Ag3) based on surface plasmon resonance (SPR)-based measurement. Prior to mixing with Ag3, the mAb3 sample was first digested by IdeS and subjected to native SEC-MS analysis to verify the digestion products. As expected, the digested sample was shown to consist of F(ab′)₂ and Fc (consists of two non-covalently interacting Fc/2) fragments that were chromatographically resolved based on their size differences (FIG. 5A). F(ab′)₂ variants with an additional N-linked glycan (mainly G2FS and G2FS2, FIG. 5A inset) were identified in a distinct SEC peak eluting before the unmodified F(ab′)₂ species. This change in SEC retention behavior is likely due to the altered protein surface characteristics (e.g., charge and/or hydrophobicity) as a result of the CDR N-glycosylation, and subsequent changes in secondary interaction with the SEC column. To assess the impact of this CDR N-glycosylation on target binding, the IdeS-digested mAb3 samples were mixed with Ag3 at various Ag-to-Ab ratios (1:2, 2:3, 1:1, 2:1, and 4:1) and then subjected to SEC-PCD-MS analysis. The SEC-UV traces of the mixture samples all displayed a distinct F(ab′)₂-Ag3 complex peak (5.1 min) that was well separated from the unbound species (FIG. 5B). As more Ag3 were introduced, there was an apparent decrease in the peak intensity of the unbound F(ab′)₂ species (6.1-7.1 min) relative to the peak intensity of the Fc fragments (7.2-7.4 min). In particular, when the Ag-to-Ab ratio rose above 1:1, no discernible UV peak corresponding to either unbound F(ab′)₂ species was detected, suggesting both unmodified and CDR N-glycosylated F(ab′)₂ species were bound to Ag3 under these experimental conditions. This observation indicated that this CDR N-glycosylation did not entirely abolish its target binding affinity, where the complex could still be formed and preserved under SEC conditions. However, close examination of the two unbound F(ab′)₂ UV peaks revealed a notably faster reduction of the unmodified species compared to the N-glycosylated species with the addition of Ag3 (FIG. 5B). This difference suggested the N-glycosylated species may have a lower target binding affinity than the unmodified species.

Alternatively, this difference in binding affinity can be more clearly demonstrated by generating the XICs of the unmodified (FIG. 5C) and N-glycosylated F(ab′)₂ species (FIG. 5D) from the PCD-MS analyses followed by comparing their corresponding distributions in the complex and the unbound forms in each mixture sample. It is evident that when Ag3 was at insufficient levels (e.g., 1:2 or 2:3), only the unmodified F(ab′)₂ exhibited binding to Ag3, while the N-glycosylated F(ab′)₂ species were entirely present in the unbound form (FIG. 5C and FIG. 5D, blue and cyan traces). At the Ag-to-Ab ratio of 1:1, virtually all the unmodified F(ab′)₂ species were transitioned into the complex form (FIG. 5C, green trace). The N-glycosylated F(ab′)₂ species started to show binding to Ag3 and were detected in both the complex and the unbound forms in the same sample (FIG. 5D, green trace). When the Ag-to-Ab ratio rose above 1:1, the N-glycosylated F(ab′)₂ species exhibited further transition into the complex form and was nearly depleted from the unbound form in the 4:1 mixture (FIG. 5D, orange and red traces). Additionally, as demonstrated by the XICs, the complexes formed by Ag3 and the unmodified F(ab′)₂ were eluted as a discrete peak (4.9-5.2 min; FIG. 5C) in all mixture samples, suggesting the complexes were stable during SEC separation.

In contrast, XICs of the N-glycosylated F(ab′)₂ species from the 1:1, 2:1 and 4:1 mixtures all displayed two broad peaks in the complex-eluting region (4.9-6.3 min; FIG. 5D, green, orange, and red traces). This likely indicated the on-column dissociation of the complexes formed by Ag3 and the N-glycosylated F(ab′)₂ during SEC separation, due to a decrease in binding affinity. These observations agreed well with a separate SPR-based affinity measurement performed using SEC-enriched samples, which detected a 2-fold increase in K_{D} from the unmodified to the CDR N-glycosylated mAb3 species. This consistency further validated the effectiveness of the affinity-resolved SEC-MS method in studying the attribute-and-function relationship. Asn55 N-glycosylation in HC CDR2 resulted in a partial loss in target binding affinity of mAb3 and thus should be considered as a CQA.

### Example 4. Evaluation of a CDR Oxidation in a Monospecific Antibody by Affinity-Resolved SEC-MS

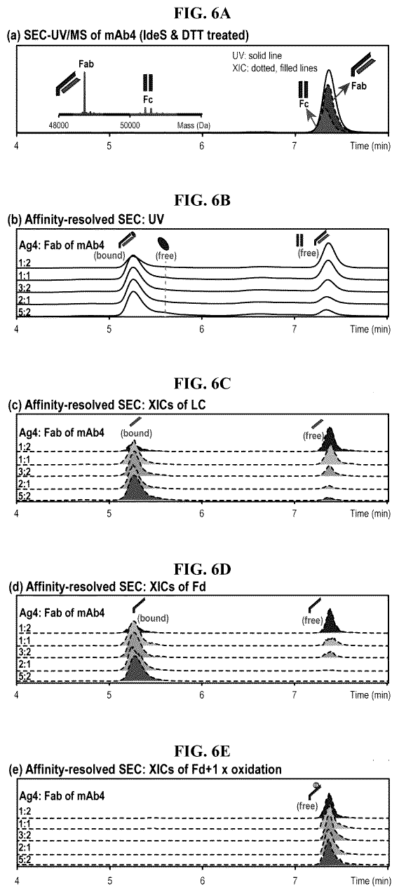

The affinity-resolved SEC-MS method was employed to evaluate the impact of a CDR oxidation (HC Met105) on target binding in a msAb (mAb4). As previously discussed, msAb contains two identical Fab arms that can potentially lead to the formation of a variety of complexes in the presence of its antigen, thus complicating the result interpretation. Therefore, they are pre-treated to generate monovalent half molecules prior to affinity-resolved SEC-MS analysis. In this example, mAb4 was first treated with IdeS digestion followed by limited reduction and alkylation to produce a mixture containing Fab and Fc fragments. Subsequent SEC-MS analysis showed that the Fab and Fc species were not chromatographically separated due to their similar sizes. However, they can be readily differentiated through intact mass measurements (FIG. 6A).

The pre-treated mAb4 sample was mixed with its target antigen (referred to as Ag4) at Ag-to-Fab ratios of 1:2, 1:1, 3:2, 2:1, and 5:2, respectively, followed by SEC-PCD-MS analysis. As demonstrated by the SEC-UV chromatograms (FIG. 6B), as more Ag4 was introduced into the mixtures, there was a notable increase in the peak intensity of the complex species (Fab-Ag4, 5.1-5.5 min) and a corresponding decrease in the peak intensity of the unbound species (unbound Fab & Fc, 7.2-7.5 min). Additionally, an apparent UV peak corresponding to the free Ag4 (5.5-5.8 min) was detected in the 5:2 mixture sample, suggesting Ag4 was in excess In this sample, a significant UV peak corresponding to the unbound species remained, which was mostly attributed to the Fc fragments. In this instance, due to the coelution of Fab and Fc, it is infeasible to rely on SEC-UV to detect potential Fab variants that had lost target binding affinity. Instead, PCD-MS data could be utilized to generate the XICs that are specific to both the unmodified and oxidized Fab species, allowing a comparison of their distributions in the complex and the unbound forms.

Due to the absence of inter-chain disulfide bond, the non-covalently associated Fab fragments were further dissociated into Fd and LC under PCD conditions prior to MS detection. In this case, the unmodified Fab was dissociated and detected as LC and Fd, whereas the oxidized Fab was dissociated and detected as LC and oxidized Fd. By generating the XICs of the Fd and its oxidized form from PCD-MS analysis, the distributions of both the unmodified and oxidized Fab species can be monitored separately. The XICs of the LC could be used to represent the overall distribution of all Fab species. As shown in FIG. 6C, the XICs of the LC showed a progressive shift in peak intensity from the unbound peak to the complex peak, as more Ag4 were introduced. However, the 5:2 mixture sample displayed a LC distribution highly similar to that of the 2:1 mixture sample, with both showing a notable unbound peak. Given the excess levels of Ag4 in these two samples, this observation implied the presence of a Fab variant with diminished target binding affinity.

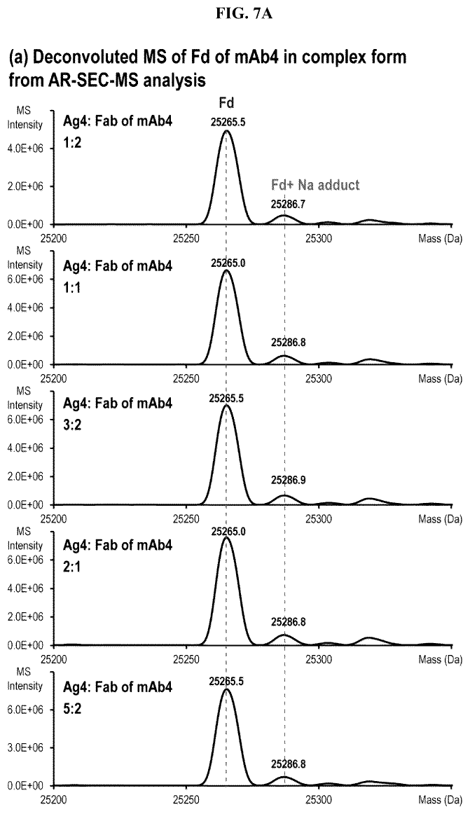

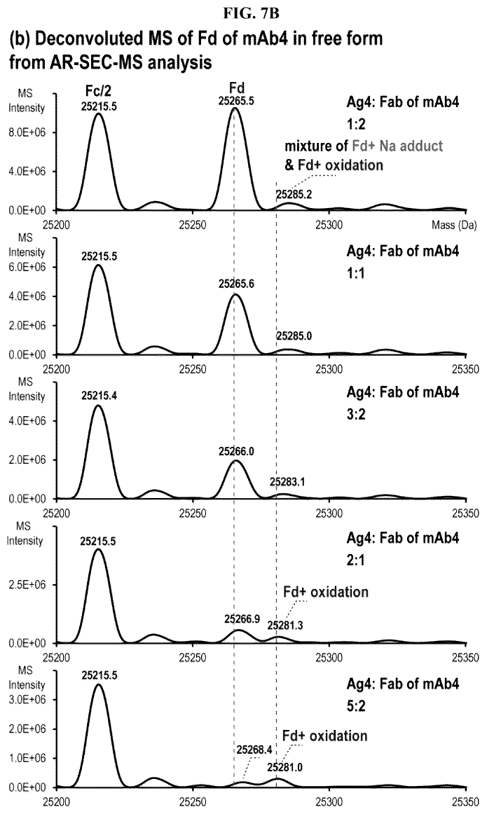

The XICs of the unmodified Fd showed a similar transition in peak intensity from the unbound peak to the complex peak as the Ag-to-Fab ratio increased from 1:2 to 2:1. The unbound peak was not detectible from the XIC of the unmodified Fd in the 5:2 mixture sample (FIG. 6D), suggesting that all the unmodified Fab species were in the complex form when Ag4 was in excess. The XICs of the oxidized Fd consistently exhibited only a single unbound peak in all the mixture samples (FIG. 6E), even with excess levels of Ag4. The deconvoluted mass spectra of Fd species from both the complex and the unbound peaks also showed the progressive enrichment of the oxidized Fd species in the unbound peak with the addition of Ag4 (FIGS. 7A and 7B). This observation suggested that this CDR Met oxidation considerably reduced, or even abolished, the target binding affinity of mAb4. The findings from this affinity-resolved SEC-MS analysis also aligned well with SPR-based affinity measurements, which reported a K_{D} of 0.3 nM (to Ag4) for the unmodified mAb4 but no binding for the Met105 oxidized form. For SPR-based affinity measurements, the oxidized variants were prepared through forced oxidation using peroxide-based reagents. This process often generates very high levels of oxidation on multiple Met sites, which may not accurately represent the variants found in a typical mAb product. Therefore, this affinity-resolved SEC-MS method is a valuable addition to conventional approaches, enabling direct assessment of potential CQAs from unenriched and undisturbed samples.

### Example 5. Study of Mutations within the Fc Region on FcRn Binding by Affinity-Resolved SEC-MS

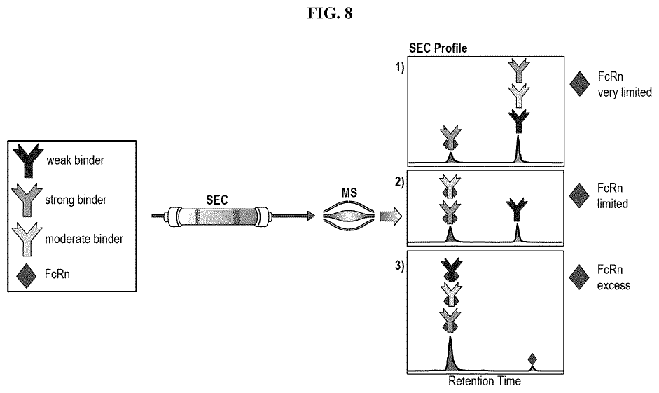

To assess the impact of mAb attributes on FcRn binding affinity, protein mixtures containing equal molars of mAbs and FcRn receptor at varying ratios and subjected to SEC-PCD-MS analysis. Under competitive binding conditions, only mAbs with the strongest affinity can bind to FcRn when FcRn is very limited in the mixture (FIG. 8). As the amount of FcRn receptor increases, more mAbs bind to FcRn, and when in excess, all mAbs can bind to FcRn, regardless of their binding strength.

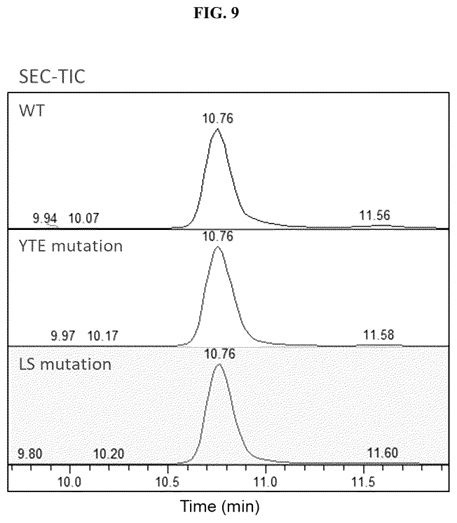

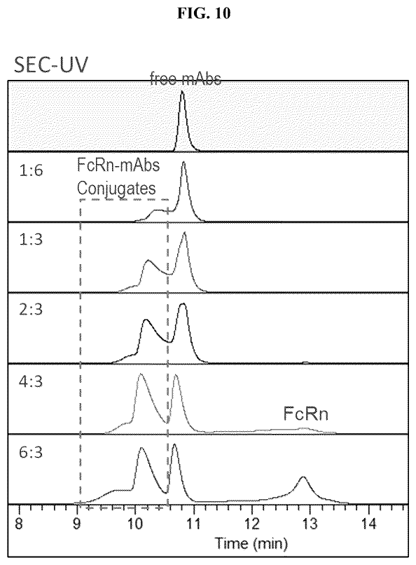

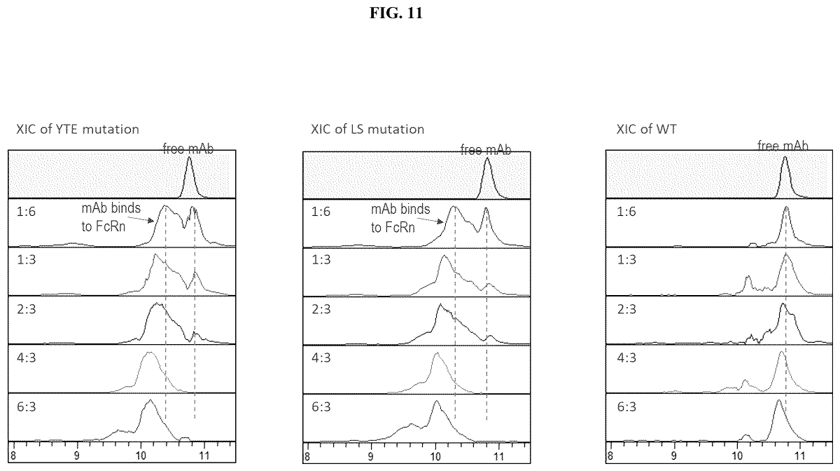

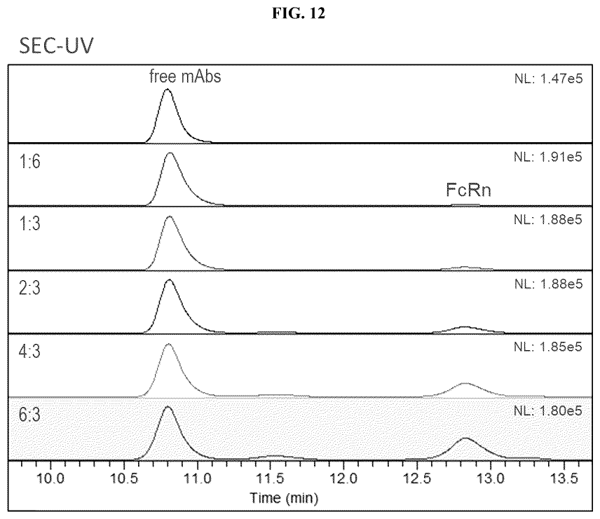

As a proof-of-concept study, an IgG1 antibody, mAb-A (wild type) and its corresponding Fc mutated forms, mAb-A^{YTE} (M255Y/S257T/T259E) and mAb-A^{LS} (M431L/N437S) were produced in house at Regeneron and compared in their FcRn affinities using the newly developed AR-SEC-MS method. Prior to the analysis, mAb-A, mAb-A^{YTE}, and mAb-A^{LS} were first mixed at equal molar ratios to generate a mAb mixture. The mixture was then mixed with FcRn at various FcRn-to-mAb molar ratios (1:6, 1:3, 2:3, 4:3, and 6:3) under two pH conditions, 5.9 and 7.3, respectively, followed by SEC-PCD-MS analysis under similar pH conditions. Comparison of the three mAbs after native SEC-MS at pH 5.9 shows the three mAbs have the same retention time, exhibiting similar masses. FIG. 9. By analyzing a series of mixtures at varying ratios of FcRn to mAbA, a corresponding increase in the formation of FcRn-mAb-A conjugates was observed. FIG. 10. A comparison of the two mutations to the wild-type mAb-A is shown in FIG. 11. Both mAb-A^{YTE} and mAb-A^{LS} exhibited an increased affinity for the FcRn receptor as observed by the formation of FcRn-mAb conjugates at the 1:6 ratio. At the elevated pH of 7.3, no retention time shift was observed for the parent mAb, indicating FcRn has weak or no affinity at this pH, as shown in FIG. 12. As FcRn only binds to IgG (Fc) at acidic pH (pH<6.5) but not at neutral pH (pH ˜7.4), the acidic operating pH is necessary for the binding analysis.

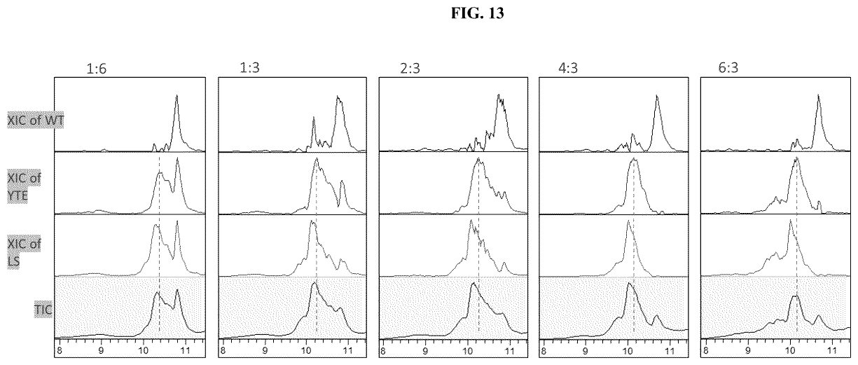

When analyzed under pH 5.8, as the FcRn-to-mAb ratio increased from insufficient to excess levels, a mAb-FcRn complex peak was observed to increase in abundance, while the unbound mAb peak displayed a corresponding decrease in abundance. This suggests the success of using SEC to monitor the FcRn interaction with mAb at pH 5.8. Using online PCD-MS detection, the distributions of mAb molecules in the complex and the unbound forms can be readily visualized through XICs. The total ion chromatogram and extracted ion chromatograms of the wild type, LS and YTE mutations is shown in FIG. 13. Specifically, XICs of each mAb revealed that, when FcRn was insufficient, both mAb-A^{YTE} and mAb-A^{LS} showed transition to the bound peak while mAb-A remained in the unbound form (FIG. 13). It was only when excessive amount of FcRn was present and that both mAb-A^{YTE} and mAb-A^{LS} were fully transitioned to the bound form, that mAb-A started to display weak binding to the FcRn. These findings indicated that YTE and LS mutation similarly enhanced FcRn affinity than the wild type Fc.

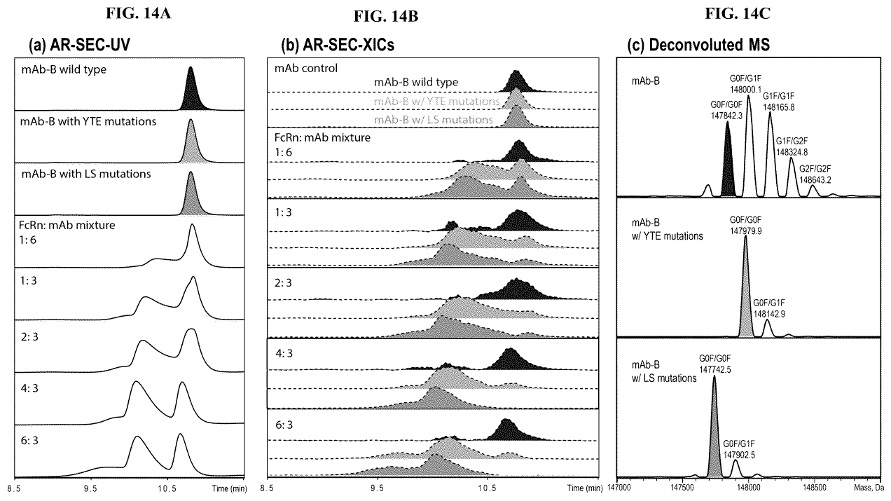

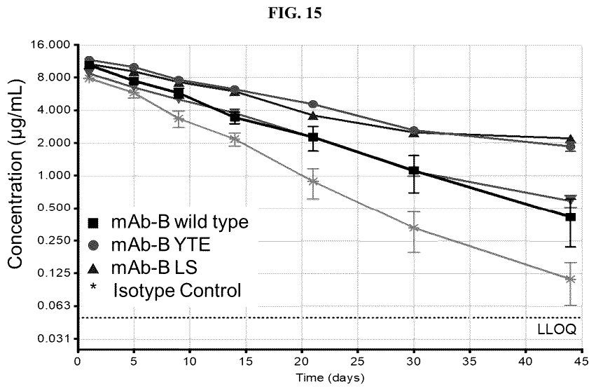

A study of the influence of Fc mutations on mAb binding to FcRn was performed. When FcRn was insufficient (1:6, 1;3 and 2:3), only YTE and LS mutants showed binding to FcRn. (FIGS. 14A and 14C). When FcRn was in excess (4:3 and 6:3) and both YTE and LS mutants were fully transitioned into the bound form, and wild type mAb-B started to form complex with FcRn. (FIGS. 14A and 14B). This complex eluted at a significantly later retention time window than the other complexes, possibly due to on-column dissociation of the weakly associated complex (dissociated constituents moved at a slower pace than the original complex due to their smaller sizes). mAbs with YTE and LS mutations showed comparable profiles in all ratios. mAbs with YTE and LS mutations showed similar affinity to FcRn at pH 5.9, which were notably higher than the affinity to FcRn of wild type mAb-B at pH 5.9. mAb-B YTE and LS Fc mutations exhibited enhanced PK over wild type mAb-B, with ˜5-6.5 fold higher drug concentrations at Day 44 (FIG. 15).

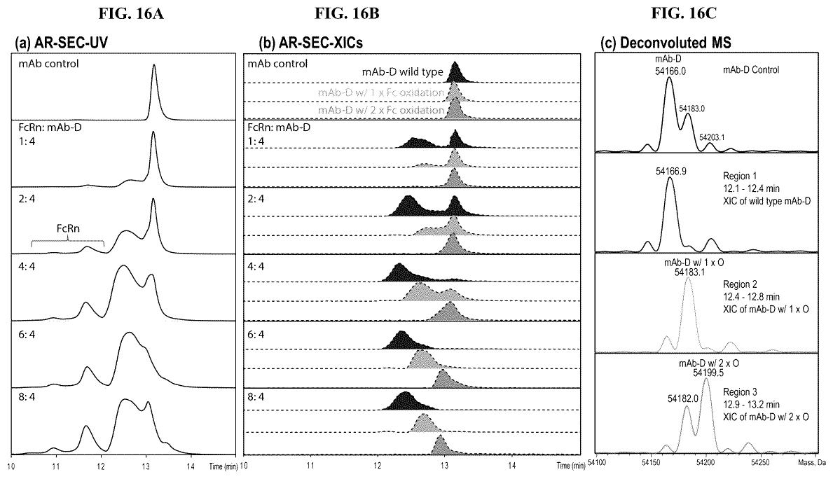

A study of the influence of Met252 oxidation on Fc binding to FcRn was performed. AR-SEC-MS demonstrated that wild type Fc showed the highest binding affinity to FcRn, transitioning first to the complex form (FIG. 16B, blue). Fc species with 1 oxidation bound to FcRn in mixtures with less affinity compared to wild type Fc (FIG. 16B, cyan). In contrast, the Fc species with 2 oxidations only bound to the FcRn in mixtures with excess receptors (FIG. 16B, green).

In summary, an affinity-resolved SEC-MS method was developed that facilitates fast and direct assessment of mAb attributes for their impact on target or Fc receptor binding. By generating varying ratios of Ag and mAb (or Fc receptor and mAb) mixtures and examining them using SEC-PCD-MS, mAb variants with reduced binding affinity were readily observed. This method does not require prior attribute enrichment through fractionation or forced degradation, making it suited for CQA evaluation during early-stage drug developability assessment.

### Example 6. Evaluation of CDR N-Glycosylation in a Bispecific Antibody (bsAb)

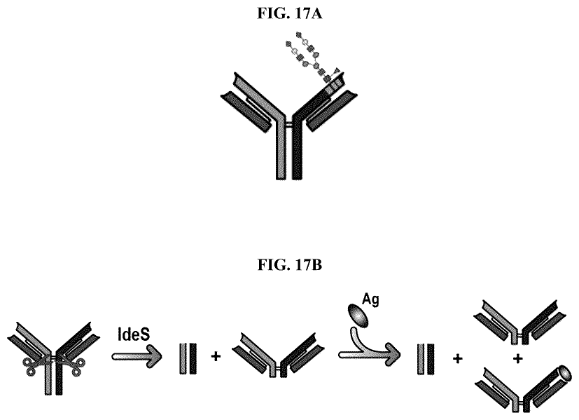

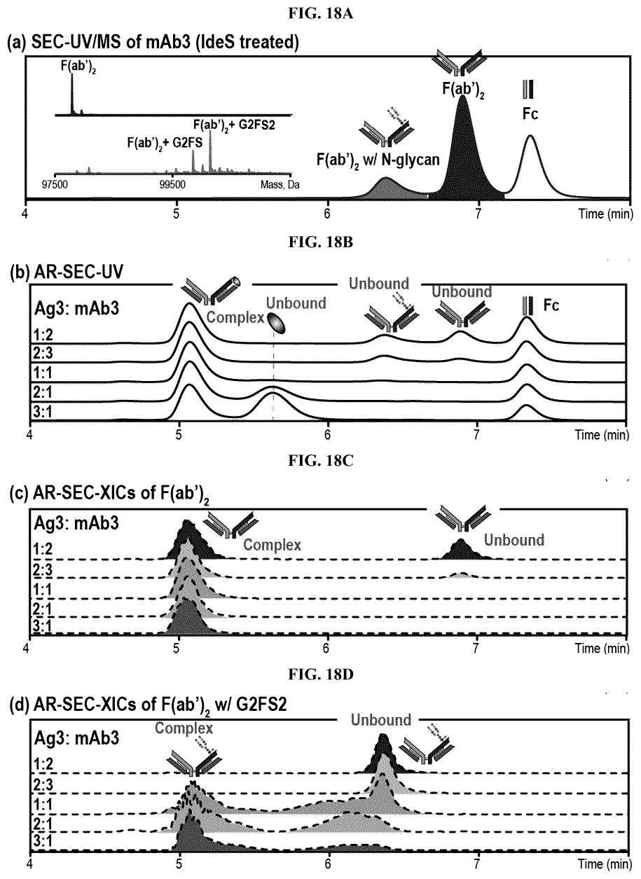

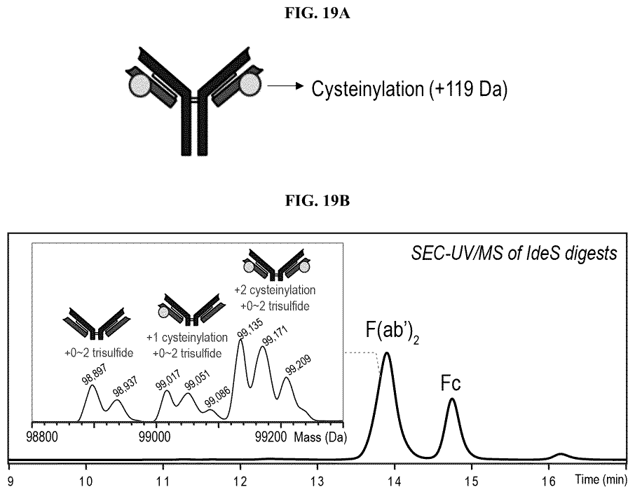

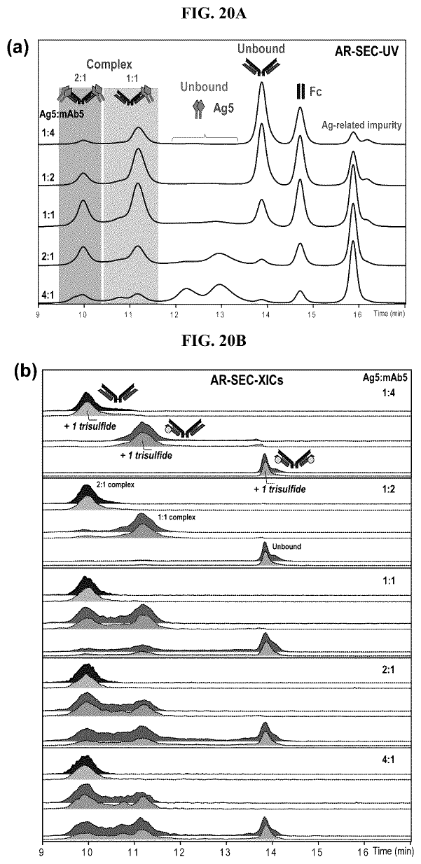

A bispecific antibody containing N-glycosylation (G2FS and G2FS2) on one of the HC CDR2 (FIG. 17A). The CDR N-glycosylated variants were separated by SEC and SPR measurement of the fractionated material showed a 2-fold increase in K_{D} (˜8 nM) as compared to an unmodified control (˜4 nM) (FIG. 18A). Prior to AR-SEC-MS analysis, the sample was first digested by IdeS to improve MS sensitivity by removing mass heterogeneity from Fc N-glycosylation as well as to improve SEC resolution by reducing the size of the analytes (FIG. 17B). Analysis by AR-SEC-UV indicated both Fab₂ forms were able to bind to the target (FIG. 18B). It was observed that the unmodified Fab₂ formed complexes first, and the complexes were stable (FIG. 18C). It was observed that the N-glycosylated Fab₂ only formed complexes after the unmodified Fab₂, and the complexes showed on-column dissociation (FIG. 18D). These data suggest that CDR N-glycosylation negatively impacts target binding.

### Example 7. Evaluation of CDR Cys Cysteinylation in a Monospecific Antibody by Affinity-Resolved SEC-MS (AR-SEC-MS)

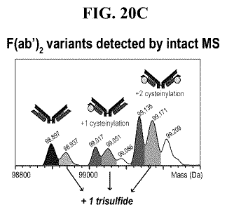

Monospecific antibody, mAb5, contains an unpaired Cys residue in LC CDR2, which was found to be extensively cysteinylated (resulting in mass increase by 119 Da) during production. Additionally, high levels of trisulfide bond (resulting in mass increase by 32 Da) were also observed at the interchain disulfide bond formed between HC and LC. These two modifications result in a mixture of mAb5 proteoforms that can be differentiated by intact mass analysis. Prior to AR-SEC-MS analysis, the mAb5 sample was first digested by IdeS and subjected to native SEC-MS analysis to verify the digestion products. The digested sample was shown to consist of F(ab′)₂ and Fc (consists of two non-covalently interacting Fc/2) fragments that were chromatographically resolved based on their size differences (FIG. 19B). From native intact mass analysis, multiple F(ab′)₂ variants contributed by the combinations of 0-2 cysteinylation and 0-2 trisulfide were detected in the mAb5 sample (inset of FIG. 19B). The AR-SEC-MS method was then employed to evaluate the impact of LC CDR2 cysteinylation on target binding affinity in mAb5. The IdeS-digested mAb5 samples were mixed with Ag5 at various Ag-to-Ab ratios (1:4, 1:2, 1:1, 2:1, and 4:1) and then subjected to SEC-PCD-MS analysis (FIG. 20A-20C). Due to the bivalent nature of mAb5, the F(ab′)₂ is able to form either a 1-to-1 or 1-to-2 complex with Ag5. As a result, the SEC-UV traces of the mixture samples displayed the detection and separation of the two complex species and the unbound species (FIG. 20A). As more Ag5 was introduced into the mixtures, there was a notable increase in the peak intensity of the complex species (9.5-11.6 min) and a corresponding decrease in the peak intensity of the unbound F(ab′)₂ species (13.7-14.1 min). To investigate the differences in binding affinity of the different F(ab′)₂ variants, XICs of the F(ab′)₂ with 0-2 CDR cysteinylation and 0-2 trisulfides were generated from the PCD-MS analyses and their corresponding distribution in the complex and unbound forms in each mixture sample were compared (FIG. 20B). When Ag5 was at insufficient levels (e.g., 1:4 or 1:2), only the unmodified F(ab′)₂ formed 1:2 complexes with the target, while the F(ab′)₂ species containing one CDR cysteinylation (on one Fab) formed exclusively 1:1 complexes, and F(ab′)₂ species with both Fab arms modified by cysteinylation remained unbound. This observation suggests that Ag preferentially binds to the unmodified Fab arm than the Fab arm modified with cysteinylation, likely due to weaker binding affinity of the latter. When the amount of Ag was further increased, and that all the unmodified F(ab′)₂ species were transitioned into the 1:2 complex form, the F(ab′)₂ cysteinylation variants began to exhibit more extensive binding with Ag5 through the Fab arm with CDR cysteinylation. Specifically, at Ag-to-Ab mixing ratios of 1:1 and above, both the F(ab′)₂+1 cysteinylation species and F(ab′)₂+2 cysteinylation species were found to form both 1:1 and 1:2 complexes with the Ag. This observation suggests that this CDR cysteinylation modification, despite weakening its target binding affinity, did not entirely abolish the binding between mAb5 and its Ag, where the complex could still be formed and preserved under SEC conditions. Interestingly, the binding behavior of each unmodified or cysteinylation-modified F(ab′)₂ species seem to be unaffected by the trisulfide modification, suggesting the interchain trisulfide formed between HC and LC does not alter the target binding affinity of mAb5.

As shown in FIG. 20B-20C, as the Ag amount increased, F(ab′)₂ without cysteinylation preferably formed 2:1 complexes, and the complexes were stable, F(ab′)₂ with 1×cysteinylation initially formed 1:1 complexes, and then only formed 2:1 complexes after the unmodified F(ab′)₂, and F(ab′)₂ with 2×cysteinylations formed both complexes last, and the complexes showed on column dissociation. These data suggest that LC CDR cysteinylation impacts target binding affinity, while inter HC/LC trisulfide bond does not.

### Example 8. Evaluation of CDR Met Oxidation in a Monospecific Antibody

An antibody containing level of Methionine (Met) oxidation (+16 Da) on HC CDR3 was evaluated. Without wishing to be bound by theory, this oxidation site is the only such site in Fd fragments in unstressed material. It was observed that this oxidation increased from ˜3% to ˜8% over a 30-month storage period at 5° C. When analyzed by SPR measurement of the peroxide-stressed material, no binding with the target was observed (Table 1).

TABLE 1

| | | PTM Percentage | | | | | |
|---|---|---|---|---|---|---|---|
| PTMs | Site | 45° C. | | | 5° C. | | |
| Name | Location | D0 | D14 | D28 | D0 | 12 Mon | 30 Mon |
| Met | HC Met¹⁰⁵ | 2.7% | 7.2% | 11.9% | 2.7% | 5.8% | 8.3% |
| Oxidation | HC Met²⁵² | 3.4% | 4.5% | 5.3% | 3.4% | 4.0% | 4.7% |
| | HC Met³⁵⁸ | 0.6% | 0.9% | 1.1% | 0.6% | 0.7% | 0.8% |
| | HC Met⁴²⁸ | 1.0% | 1.4% | 1.7% | 1.0% | 1.4% | 1.6% |
| | LC Met⁴ | 0.1% | 0.1% | 0.1% | 0.1% | 0.1% | 0.1% |

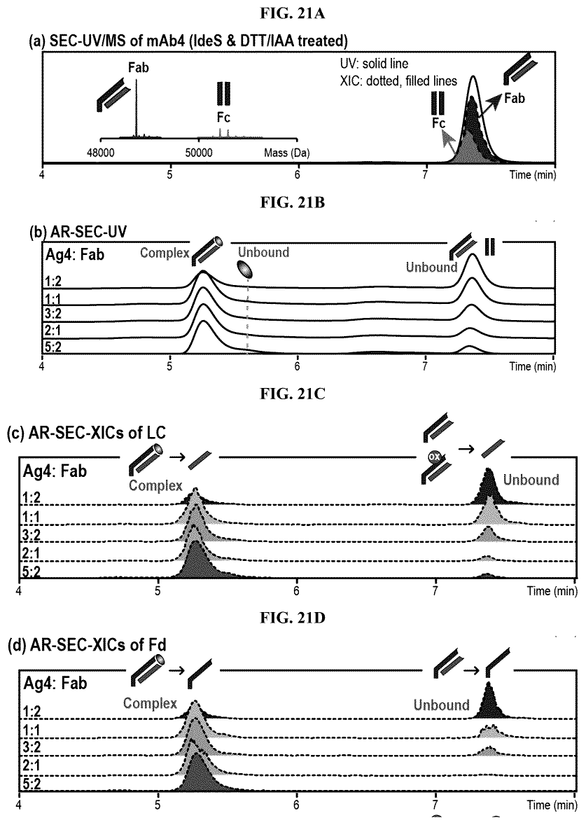

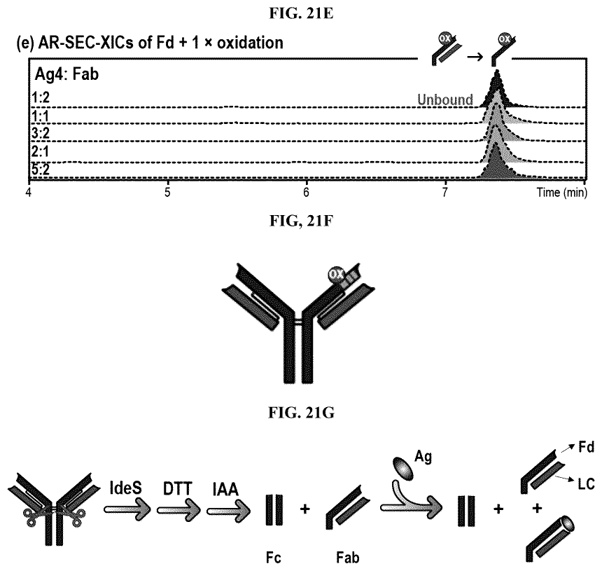

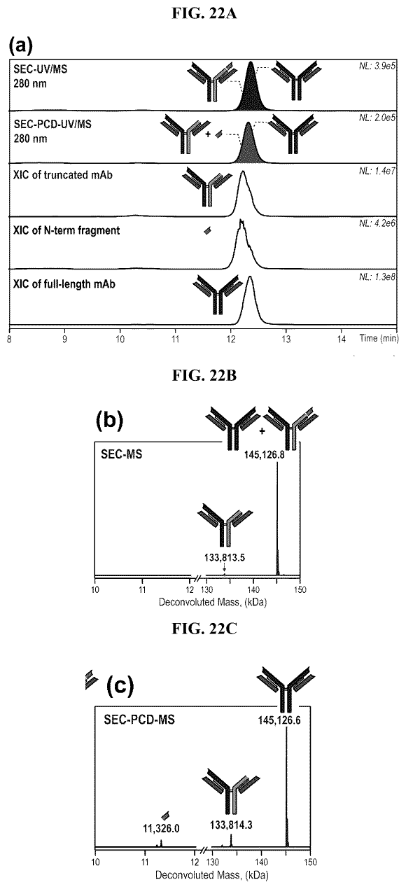

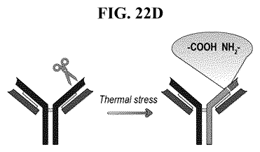

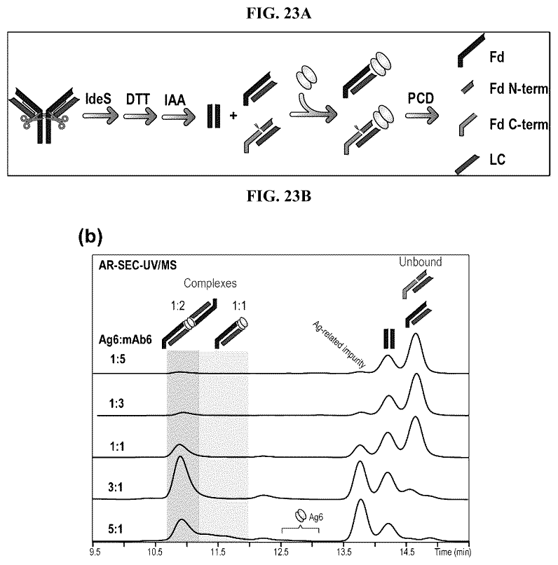

Prior to AR-SEC-MS analysis, the sample was first digested by IdeS, DTT, and IAA to simplify the immunocomplex heterogeneity due to various binding stoichiometries and improve MS sensitivity and SEC resolution. PCD further dissociated the Fab into Fd and LC fragments, facilitating the detection of PTMs with small mass changes and their localization (FIG. 21G). In samples with an excess of Ag, notable levels of LC signal were still detected in the unbound form, suggesting a subpopulation of Fab was unable to bind to the Ag (FIG. 21C). It was observed that unmodified Fd signal was depleted from the unbound form in samples with an excess of Ag (FIG. 21D). It was also observed that oxidized Fd signal was only found in the unbound form, suggesting this CDR oxidation led to diminished Ag binding affinity (FIG. 21E).

### Example 9. Evaluation of CDR Clipping in a Monospecific Antibody (msAb) by Affinity-Resolved SEC-MS (AR-SEC-MS)

Monospecific antibody, mAb6, was found to develop a notable level of site-specific CDR3 clipping after thermal stress (40° C.) (FIG. 22D). This CDR3 clipped species remained intact under native buffer conditions due to the non-covalent interactions between VH and VL domains. To detect and characterize the CDR3 clipped species, mAb6 sample stressed under 40° C. for 1 month was subjected to native SEC-UV/MS and SEC-PCD-UV/MS analyses (FIG. 22A-22C). The SEC-UV traces revealed only one predominant peak, which corresponds to the intact mAb6 (FIG. 22A, red and blue traces). These peaks, when detected under native conditions, were found to display one predominant mass peak at 145,126.8 Da (FIG. 22B), which agrees with the intact mass of mAb6. Additionally, a minor mass peak at 133,813.5 Da was also detected, which corresponds to the C-term fragment of the clipped mAb6 and was likely induced by in-source dissociation of the clipped mAb6. Due to the small delta mass introduced by clipping reaction (+18 Da), the unclipped mAb6 and clipped mAb6 cannot be differentiated by intact mass measurements. Therefore, the experimental intact mass obtained under native conditions likely represents a mixture of the clipped and unclipped mAb6. Alternatively, when the intact mass detection was performed under denaturing conditions (SEC-PCD-MS), three mass species were detected, representing the unclipped mAb6 (145,126.6 Da), C-term fragment of clipped mAb6 (133,814.3 Da), and N-term fragment of clipped mAb6 (11,326.0 Da) (FIG. 22C). Further, using their corresponding XICs, the elution profiles of the three species were reconstructed (FIG. 22A). The XICs revealed highly similar elution profiles of the N-term and C-term fragments of clipped mAb6, which eluted slightly earlier than the unclipped mAb6. Together, these results successfully detected the HC CDR3 clipped species in the stressed mAb6 sample and confirmed its “intact” structure maintained by non-covalent VH-VL interactions under native conditions.

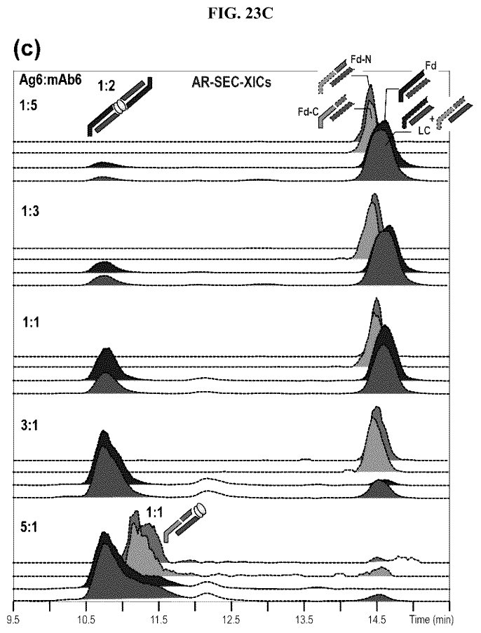

To evaluate the impact of CDR3 clipping on target binding of mAb6, the affinity-resolved SEC-MS method was applied. Ag6, the binding target of mAb6, was produced as a covalent dimer that can bind to two mAb6 molecules at its max capacity. Considering the bivalent nature of both mAb6 and Ag6, their interactions can lead to a highly complex profile of immunocomplexes, ranging from 1:1 binding complex to larger aggregates, and hence complicate the result interpretation. Therefore, mAb6 was first pre-treated with IdeS digestion followed by limited reduction and alkylation to generate monovalent half molecules, producing a mixture containing Fab and Fc fragments (FIG. 23A). Next, the pre-treated mAb6 sample was mixed with Ag6 at Ag-to-Ab ratios of 1:5, 1:3, 1:1, 3:1, and 5:1, respectively, followed by SEC-PCD-MS analysis. As demonstrated by the SEC-UV chromatograms (FIG. 23B), as more Ag6 was introduced into the mixtures, there was a notable increase in the peak intensity of the complex species (10.6-12.0 min) and a corresponding decrease in the peak intensity of the unbound Fab species (14.5-15 min). The complexes formed between Ag6 and Fab can adopt both a 1:2 (10.6-11.2 min) and 1:1 (11.2-12 min) stoichiometry. Comparisons of the distribution of CDR unclipped and clipped Fab species in the complex and the unbound forms were conducted. Due to the absence of interchain disulfide bonds, the non-covalently associated Fab fragments were further dissociated into Fd and LC under PCD conditions prior to MS detection. In this case, the unclipped Fab was dissociated and detected as LC and Fd, whereas the CDR3 clipped Fab was dissociated and detected as LC, Fd N-term (fragment) and Fd C-term (fragment). Hence, by generating the XICs of the Fd, Fd N-term and Fd C-term fragments from PCD-MS analysis (FIG. 23C), the distributions of both the unclipped and CDR3 clipped Fab species were monitored separately. Meanwhile, the XICs of the LC could be used to represent the overall distribution of all Fab species. As shown in FIG. 23C, the XICs of the LC (red filled trace) showed a progressive shift in peak intensity from the unbound peak to the complex peak, as more Ag6 were introduced. The XICs of the intact Fd (blue filled trace) showed a similar transition in peak intensity from the unbound peak to the complex peak as the Ag-to-Ab ratio increased. In addition, the unbound peak was not detectible from the XIC of the intact Fd in the 5:1 mixture sample (FIG. 23C), suggesting that all the unclipped Fab species were in the complex form. In contrast, the XICs of the N-term and C-term fragments of Fd (magenta and green, respectively) both exhibited a single unbound peak in the mixture samples containing insufficient amount of Ag6 (from 1:5 to 3:1), suggesting the clipped Fab species has a weaker target binding affinity than the unclipped form. Notably, when Ag6 is in excess (5:1 mixture) and the unclipped Fab species were entirely populated in the bound forms, the clipped Fab began to bind to Ag6, forming 1:1 complexes (FIG. 23C). This observation suggests that this CDR3 clipping reduced, but did not abolish, the target binding affinity of mAb6.

### Example 10. Evaluation of SCX-MS as a Method to Monitor CDR Modifications w/Site-Specific Resolution

Therapeutic monoclonal antibodies (mAbs) often host a large number of post-translational modifications (PTMs), which are referred to as quality attributes, that can impair the drug efficacy and/or safety. Assessment of these quality attributes is an important aspect during the development of therapeutic mAbs. This is traditionally performed in a low-throughput and laborious fashion by attribute enrichment followed by binding affinity measurements. The present study describes an AR-SEC-MS (affinity-resolved size exclusion chromatography coupled to mass spectrometry) method for fast and direct assessment of mAb attributes for their impact on target or Fc receptor binding. By generating varying ratios of Ag and mAb (or Fc receptor and mAb) mixtures and examining them using SEC-PCD-MS (post-column denaturation assisted SEC-MS), mAb variants with reduced binding affinity were readily observed. In particular, utilizing XICs from the SEC-PCD-MS analysis, the distribution of mAb variants in the complex and unbound forms can be visualized, allowing a direct comparison of their binding affinity. This AR-SEC-MS method, however, is limited in studying PTMs that are associated with minimal mass changes, such as Asn deamidation (+1 Da) and Asp isomerization (+0 Da), and is insensitive to distinguishing site-specific PTMs. This is because SEC separation is generally insensitive to PTMs (both CDR or non-CDR) and intact mass analysis alone is insufficient to confidently differentiate the protein attributes associated with small mass changes.

Herein, the inventors of the present disclosure developed a new method called 2D affinity-resolved SEC-SCX-MS (2D AR-SEC-SCX-MS), achieves separation and identification of a wide range of CDR modifications due to its excellent selectivity towards surface modifications. The method provides affinity-based evaluation of CDR PTMs (including those with minimal mass changes) by allowing separation of CDR modified mAb (or mAb subunits) from the unmodified at the chromatography scale. This allows the method to be universally applicable to analyze the attribute-and-function relationship of CDR PTMs without being restricted by the significance of mass changes associated with the specific PTM.

In the following sections, we describe in detail the workflow of the 2D SEC-SCX-MS method. Two case studies were also discussed in detail to showcase the utility of this new method in assessing the impact of CDR modifications on target binding.

Limitations of AR-SEC-MS in Studying CDR Modifications with Small Mass Changes

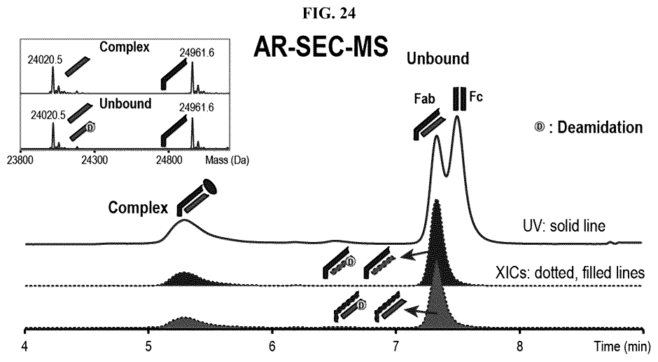

To showcase the limitations of AR-SEC-MS in studying mAb CDR modifications with small mass changes (e.g., Asn deamidation and Asp isomerization), a monospecific antibody that contains a Asn deamidation in LC CDR2, mAb1, was subjected to AR-SEC-MS analysis with its antigen, Ag1. First, mAb1 was pre-treated with IdeS digestion followed by limited reduction and alkylation to generate monovalent half molecules, producing a mixture containing Fab and Fc fragments. Next, the pre-treated mAb1 sample was mixed with Ag1 at Ag-to-Fab ratio of 1:2, followed by SEC-PCD-MS analysis (FIG. 24). SEC-UV trace shows the detection of three major peaks, each corresponding to Fab-Ag complex, unbound Fab, and Fc fragments. Notably, due to the absence of interchain disulfide bond, the non-covalently associated Fab fragments were further dissociated into Fd and LC under PCD conditions prior to MS detection. As a result, online mass measurements of the Ag-Fab complex peak and unbound Fab peak each detected two predominant mass peaks corresponding the Fd (24,961.6 Da) and LC (24020.5 Da), respectively. The Fab variant with Asn deamidation on LC CDR2, despite being present in the sample, cannot be differentiated from the unmodified Fab due to the small mass differences (1 Da). Additionally, both XICs of the Fd (blue trace, FIG. 24) and LC (red trace, FIG. 24) species monitor the collected distribution of both unmodified Fab and LC CDR deamidated Fab species in the complex and unbound forms. As a result, this method is not suitable for analysis regarding the target binding affinity differences of the unmodified Fab and LC CDR deamidated Fab species were achieved using AR-SEC-MS.

### Affinity-Resolved SEC-SCX-MS Workflow

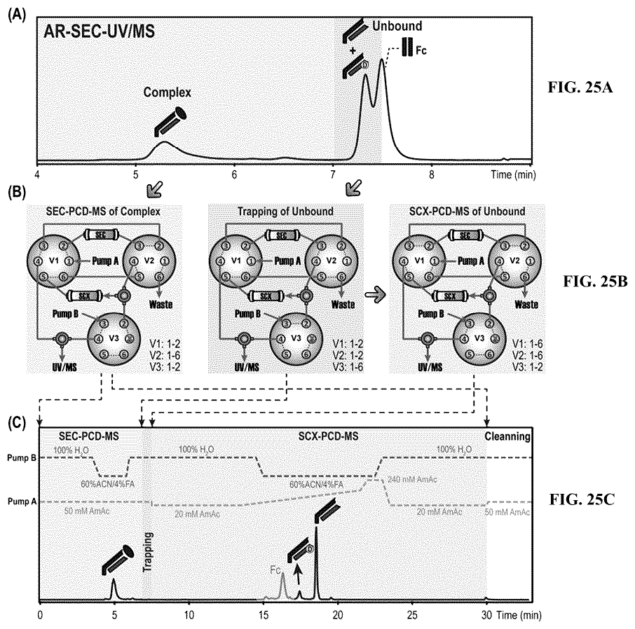

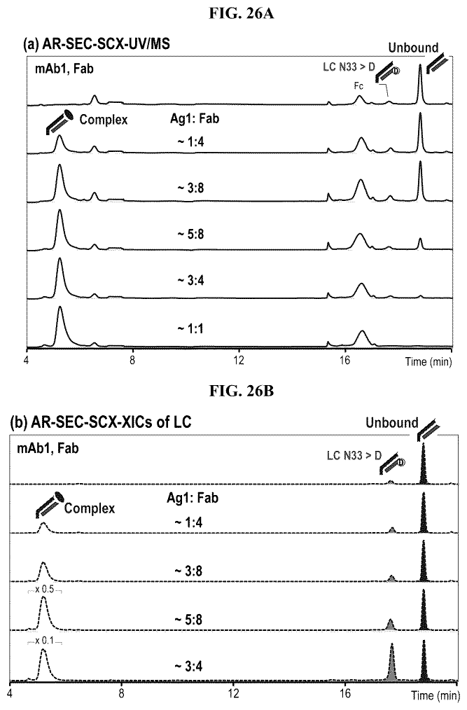

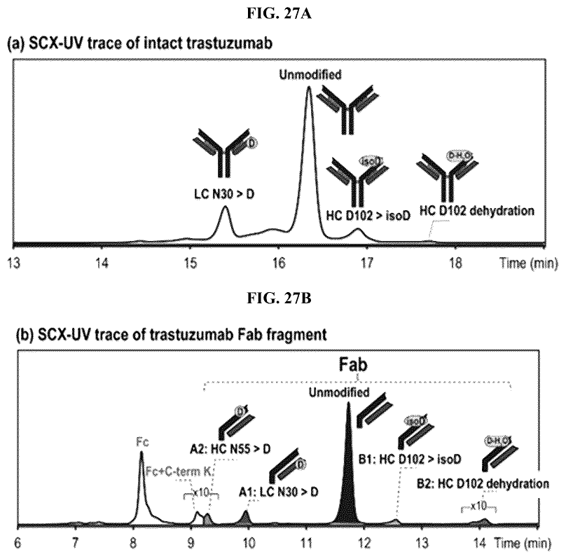

To address this unmet need, the inventors of the present disclosure prepared a series of Ag and mAb mixtures by introducing Ag to the corresponding mAb samples from insufficient to excess molar amounts and subjected to 2D SEC-SCX-MS analysis. SEC is used as the 1^{st} analytical dimension to separate the bound and unbound species based on their differences in hydrodynamic radii in each mixture. After the separation, the bound species were directly subjected to online mixing with a denaturing solvent to liberate the mAb species from the mAb-Ag complex followed by intact mass measurement (FIG. 25A). The denaturing solvent composition (60% acetonitrile and 4% formic acid) was optimized to disrupt many known non-covalent interactions (e.g., HC-LC and HC-HC interactions in partially reduced Ab and Ag-Ab complexes) instantaneously upon post-column mixing while maintaining the dissociated protein components in the solution phase for MS detection. Subsequently, the unbound species from SEC elution was subjected to an online 2^{nd} dimension strong cation exchange (SCX) chromatography (FIG. 25A), which effectively separated the CDR modified variants from the unmodified species, followed by online MS analysis with PCD enabled. As a result, the final chromatograph from the 2D AR-SEC-SCX-MS consists of two continuous segments, the first of which being SEC-PCD-MS analysis of the mAb from the bound species and the second being SCX-PCD-MS analysis of the unbound mAb. Finally, using the extracted ion chromatograms (XICs), the distributions of mAb variants in bound and unbound forms at varying mixing ratios were used to assess their relative affinities to the target. Under competitive binding conditions, mAb proteoforms with higher binding affinities (e.g., unmodified mAb) were preferentially enriched into the complex form, while the proteoforms with lower binding affinities (e.g., mAb variants) were enriched into the unbound form. Further, in cases where mass measurement is insufficient to differentiate the mAb variant form and unmodified form, by comparing their depletion rate from the unbound population (as monitored by SCX-MS) in the protein mixtures with increasing amount of Ag, their relative affinity towards the Ag was compared. It is worth noting that with PCD enabled during MS detection in 2D SEC-SCX-MS workflow, it is only necessary to monitor the mAb species by MS, allowing the often highly heterogeneous antigens or receptors to be excluded from the analysis. In addition, the employed SEC (or SCX)-PCD-MS method produces native-like spectra for mAb molecules, which greatly simplifies the data interpretation, due to the separation of mAb signal from its binding partners in the m/z space. The scheme of the 2D SEC-SCX-MS configuration design is shown in FIG. 25B. A 2D HPLC system equipped with two binary pumps and three external 6-port diverting valves are required for this configuration. In this study, a Vanquish™ Flex 2D-LC system (Thermo Fisher Scientific, Bremen, Germany) was used. A Thermo Scientific Q Exactive™ UHMIR Hybrid Quadrupole Orbitrap™ Mass Spectrometer (MS) was used for intact mass analysis. The LC and MS was interfaced using a Microflow-Nanospray Electrospray Ionization (MnESI) source and an 8-nozzle, 10 μm ID Microfabricated Monolithic Multinozzle (M3) emitter (Newomics, Berkeley, CA). When developing a 2D SEC-SCX strategy, a challenge is the mobile phase compatibility. Ammonium acetate is a MS compatible salt to substitute conventional non-volatile salts for both SEC and SCX analysis when directly coupling with MS detection. For SEC applications, ammonium acetate buffer is prepared at higher salt concentrations (50-200 mM, pH 6.8) to minimize undesired secondary interaction between the protein analytes and the column stationary phase. However, in order to successfully retain the mAb or mAb subunit species on SCX column, the loading buffer was maintained at low concentration (e.g., 20 mM of ammonium acetate) and/or slightly acidic (e.g., pH between 5 and 6). Out of these considerations of mobile phase compatibility, a SEC mobile phase consisting of 50-75 mM of ammonium acetate was selected. After SEC elution prior to SCX loading, the SEC eluent flow was mixed with a separate flow of water to lower the salt concentration to 20 mM to allow successful trapping of the protein analyte on the SCX column (FIG. 25B). Subsequently, a salt gradient (from 20 mM to 240 mM) was then applied for charge variant separation by SCX. Using the same example of mAb1, the utility of the 2D SEC-SCX-MS in differentially monitoring the unmodified Fab and LC CDR2 deamidated Fab are illustrated in FIG. 25A-25C. At the method start, an aliquot of the Ag-Fab mixture sample was injected onto the SEC column and separated using mobile phase consisting of 50 mM ammonium acetate. The first 7 min of SEC analysis (before the unbound Fab elutes) was directly routed to interface with MS (FIG. 25A-25C, left, blue shaded regions), with PCD enabled during Ag-Fab complex elution (by mixing the SEC eluent with denaturant flow prior to MS) to liberate the Fab from the complex for MS detection. During the SEC elution of unbound Fab (7-7.5 min), the valve positions were set to have SEC eluent directly connected with the inlet of SCX column, where it was mixed with a flow of water before entering the SCX column (FIG. 25A-25C, middle, grey shaded regions). During this step, the unbound Fab species were trapped on the SCX column. After loading of the unbound Fab fraction to the SCX column completed (at 7.5 min), the valve positions were immediately changed to remove SEC from the SCX flow path and allow a salt gradient to be directly applied on the SCX column for charge variant separation, elution, and MS detection of the unbound Fab species (FIG. 25B-25C, right, green shaded regions). FIG. 25C (right, green shaded region) displays the SCX-TIC of the unbound Fab of mAb1 from the mixture sample, highlighting chromatographic separation of the unmodified Fab and LC CDR2 deamidated Fab species, as well as Fc species that were introduced from the SEC fractionation step due to its partial coelution with Fab (FIG. 25A). After SCX-MS analysis completed, the valve positions were changed back to the initial condition to allow desalting and cleaning of the SEC column before the next injection (FIGS. 25A and 25C, far right, yellow shaded region).

### Example 11. Evaluation of CDR Asn Deamidation in mAb1 by 2D Affinity-Resolved SEC-SCX-MS (AR-SEC-SCX-MS)

The 2D AR-SEC-SCX-MS method was used to evaluate the impact of LC CDR2 Asn33 deamidation on target binding in mAb1. Similar to the sample preparation strategies used in AR-SEC-MS method disclosed above, the mAb1 sample was first pre-treated with IdeS digestion followed by limited reduction and alkylation to generate monovalent half molecules, producing a mixture containing Fab and Fc fragments. Next, the pre-treated mAb1 sample was mixed with Ag1 at various Ag-to-Fab ratio (1:4, 3:8, 5:8, 3:4, and 1:1, ratios are estimated based on stock protein concentrations), followed by SEC-SCX-MS analysis (FIG. 26A-26B). As discussed above, the SEC-UV traces of the mixture samples each consist of two segments, namely, the SEC segment which detected the Fab-Ag1 complex species (5-6 min) and the subsequent SCX segment that detected the unbound Fab species (including Fab variant with LC Asn33 deamidation and unmodified Fab, 17.5-19 min) and Fc fragments (FIG. 26A). As more Ag1 were introduced, there was an apparent decrease in the peak intensity of both unbound Fab species. In particular, when the Ag-to-Ab ratio increased to 1:1, no discernible UV peak corresponding to either unbound Fab species was detected, suggesting both unmodified and CDR deamidated Fab species were bound to Ag1 under these experimental conditions. This observation indicated that this CDR deamidation did not entirely abolish its target binding affinity, where the complex could still be formed and preserved under SEC conditions. However, close examination of the two unbound Fab UV peaks revealed a notably faster reduction of the unmodified species compared to the deamidated species with the addition of Ag1 (FIG. 26A). As the faster reduction in the unbound form implied faster binding to Ag1, this difference suggested the CDR deamidated species may have a lower target binding affinity than the unmodified species. Alternatively, this difference in binding affinity can be more clearly demonstrated by generating the XICs of LC from the PCD-MS analyses, which represent both the unmodified Fab (FIG. 26B, blue filled peak) and CDR deamidated Fab (FIG. 26B, orange filled peak), followed by comparing their relative intensities in the unbound fraction. Using the XIC peak area, it is evident that the relative abundance of the CDR deamidated Fab variant in the unbound fraction showed an increasing trend with the addition of more Ag1 to the sample, supporting the conclusion that LC CDR Asn33 decreased the target binding affinity of mAb1.

### Example 12. Multiplexed Evaluation of CDR Modifications in Trastuzumab

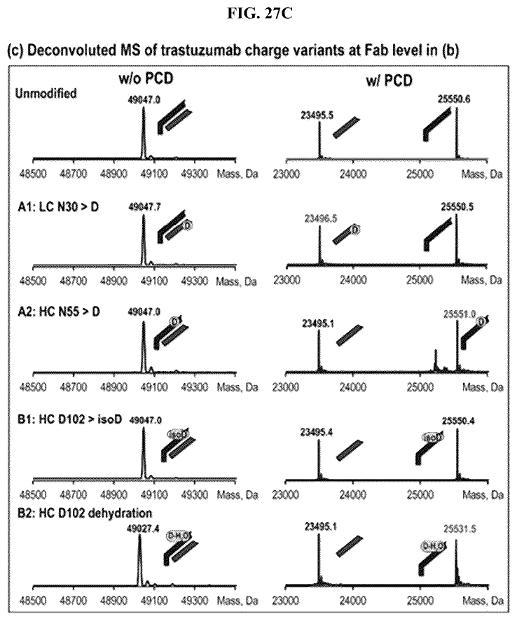

Due to the high selectivity of SCX separation towards mAb CDR modifications and high specificity of MS detection, the 2D AR-SEC-SCX-MS strategy is effective for simultaneous evaluation of multiple CDR modifications in a mAb. For example, the 2D AR-SEC-SCX-MS method was applied to study trastuzumab and the impact of different CDR modifications on this antibody. Trastuzumab is an antibody, and its unstressed form has been reported to contain four CDR modifications, including HC Asn55 deamidation, LC Asn30 deamidation, HC Asp102 isomerization, and HC Asp102 dehydration. Additionally, SCX is effective in enabling separation of these CDR-modified variant forms. To test the separation efficiency using our MS-compatible SCX chromatography method, both untreated trastuzumab and trastuzumab subunits (treated with IdeS digestion and limited reduction and alkylation to remove and alkylate inter-chain disulfide bonds) were subjected to SCX-UV/MS analysis. As a result, SCX-UV chromatogram of trastuzumab (FIG. 27A) showed effective separation of the CDR modified variants. Subsequent SCX-MS analysis of trastuzumab subunits (Fab and Fc) further revealed the successful separation of CDR modifications at the Fab level, of which five distinct charge variant peaks were of interest and identified to be Fab w/HC Asn55 deamidation, Fab w/LC Asn30 deamidation, unmodified Fab, Fab w/HC Asp102 isomerization, and Fab w/HC Asp102 dehydration (FIG. 27B). Their identification was supported by online intact mass measurement under both native conditions (w/o PCD) and denaturing conditions (w/PCD) (FIG. 27C). Further, the charge variant peaks of interest (A1, A2, main, B1, and B2, FIG. 27B) were also fractionated and subjected to enzymatic digestion and bottom-up MS analysis to confirm the location of each CDR modification. Table 2 summarized the detected PTM level in each SCX fraction, further supporting their proposed identities.

TABLE 2
Quantitation of CDR PTMs detected in SCX fractions
from IdeS, DTT, and IAA treated trastuzumab sample

| | A2 | A1 | main | B1 | B2 |
|---|---|---|---|---|---|
| LC N30 Ammonia Loss | 0.0% | 0.0% | 0.0% | 0.0% | 0.5% |
| LC N30 Deamidation | 20.3% | 92.6% | 0.4% | 0.7% | 15.0% |
| HC N55 Ammonia Loss | 1.7% | 3.2% | 2.9% | 2.8% | 1.0% |
| HC N55 Deamidation | 45.7% | 4.3% | 3.2% | 4.0% | 1.1% |
| HC D102 Isomerization | 5.4% | 4.3% | 2.7% | 71.2% | 52.4% |
| HC D102 Dehydration | 0.4% | 0.4% | 0.3% | 0.4% | 11.4% |

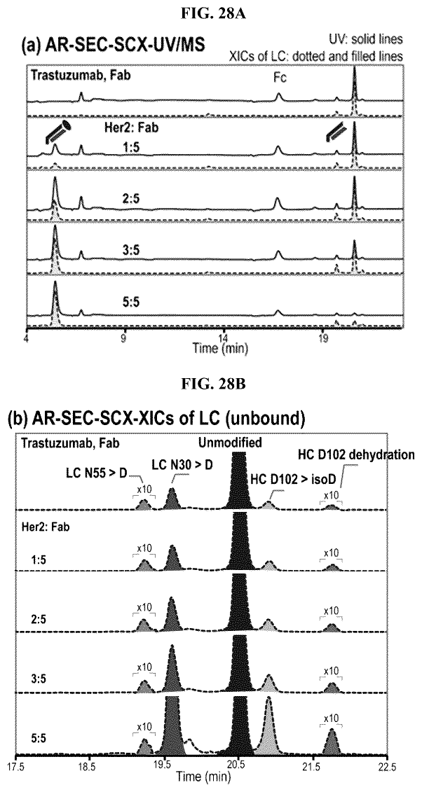

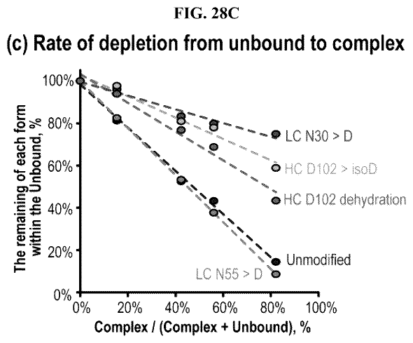

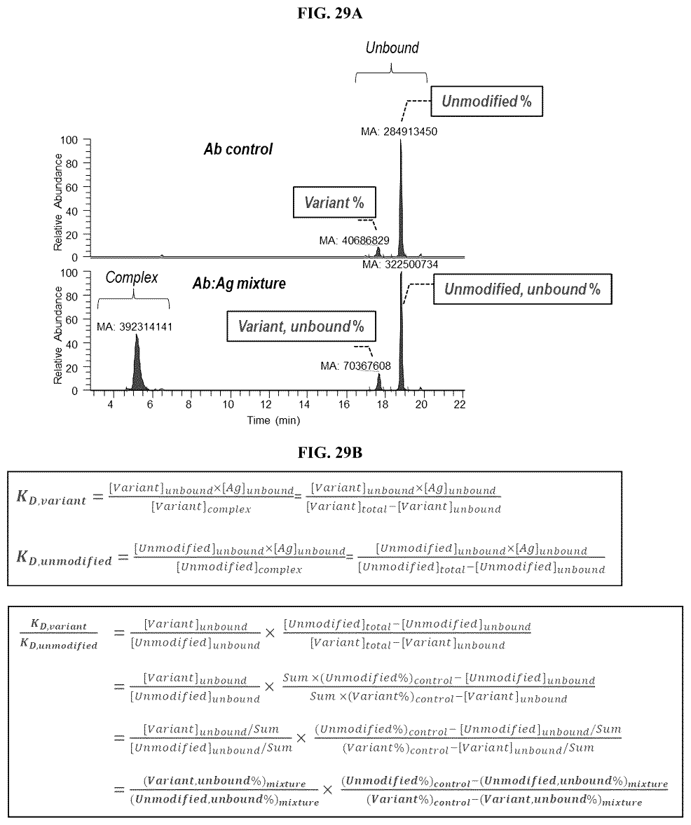

2D AR-SEC-SCX-MS was performed on trastuzumab to evaluate the impact of the above identified CDR PTMs on its target binding affinity. The trastuzumab sample was first treated with IdeS digestion followed by limited reduction and alkylation to produce a mixture containing Fab and Fc fragments. As discussed above, this treatment disrupted all inter-chain disulfide bonds and produced non-covalently associated Fab fragments, which were further dissociated into Fd and LC when subjected PCD-MS analysis. The pre-treated trastuzumab sample was then mixed with its target antigen (ectodomain of Her2) at Ag-to-Fab ratios of 1:5, 2:5, 3:5, and 5:5, respectively, followed by 2D SEC-SCX-MS analysis. As observed in the SEC-SCX-UV chromatograms (FIG. 28A), as more Her2 was introduced into the mixtures, there was a notable increase in the peak intensity of the complex species (Fab-Her2, 5-6 min) and a corresponding decrease in the peak intensity of the unbound Fab species (18.5-22.5 min). By generating the XICs of the LC from PCD-MS analyses, the relative abundance changes of the CDR modified Fab variants in the unbound population were monitored (FIG. 28A, yellow filled trace and FIG. 28B). Visual examination of the XIC peaks in the unbound Fab region (separated by SCX) revealed that most CDR modified variants showed notable enrichment in the unbound fraction compared to the unmodified form, except LC Asn55 deamidated Fab. Further, a value of “the remaining of each variant within the bound %” (value=100% for each variant in neat trastuzumab sample) from each mixture sample can be calculated using Equation 1:

$$Equation 1$$

$$\% Variant X in unbound = \frac{{Int}_{Vx - mix} / ( \sum {Int}_{unbound - mix} + {Int}_{complex - mix} )}{{Int}_{Vx - neat} / \sum {Int}_{unbound - neat}}$$

Where Variant X refers to each Fab variant (A1, A2, main, B1 and B2 from FIG. 28B), “% Variant X in unbound” refers to the “remaining of Variant X within the unbound %”. All “Int” values were generated using the peak area of XIC of LC. Intvₓ₋ₘᵢₓ refers to the intensity of Variant X in the unbound form in the mixture sample, ΣInt_{unbound-mix} refers to summarized intensity of all Fab species (A1, A2, main, B1, and B2) in the unbound fraction from the mixture sample, Int_{complex-mix} refers to the intensity of all Fab species in the complex fraction from the mixture sample, Int_{Vx-neat} refers to the intensity of Variant X in the neat trastuzumab Fab sample (without Her2), and ΣInt_{unbound-neat} refers to summarized intensity of all Fab species (including A1, A2, main, B1, and B2) in the neat trastuzumab Fab sample.

Additionally, the relative distribution of trastuzumab in the complex fraction in each mixture sample can be calculated using Equation 2:

$$\frac{complex}{complex + unbound} ( \% ) = \frac{{Int}_{complex - mix}}{( \sum {Int}_{unbound - mix} + {Int}_{complex - mix} )} \qquad Equation 2$$

By plotting the “the remaining of each variant within the bound %” against “the relative distribution of trastuzumab in the complex form” for each Fab species in each mixture sample, the depletion rates of each Fab species from the unbound to complex were compared (FIG. 28C), where a faster depletion rate indicates stronger target binding affinity and a slower depletion rate indicates a weaker target binding affinity. Consequently, the relative binding affinities of the different CDR modified variant species and unmodified species of trastuzumab were ranked as: LC N30 deamidation<HC D102 isomerization <HC D102 cyclization <LC N55 deamidation≈unmodified.

### Example 13. Quantitative Assessment of K_{D} Changes Based on 2D AR-SEC-SCX-MS Data

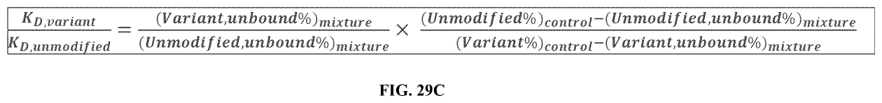

Quantitative information of different variant species measured by MS intensities (e.g., XIC peak area) from AR-SEC-SCX-MS analysis were used to enable quantitative assessment of the K_{D} changes caused by the mAb variant. Using the example of mAb1 from Example 6 above, calculation of the relative value of the K_{D} of the Fab variant (CDR deamidation) to the K_{D} of the unmodified Fab of mAb1 is shown in FIG. 29A-29C. Analyses of two samples facilitated this K_{D} comparison, including a neat Ab sample (FIG. 29A, blue filled trace) and a Ag-Ab mixture sample where Ag is insufficient (FIG. 29A, red filled trace). Using the intensity values generated from XIC (of LC) peak areas from both sample analyses and applying the fundamental K_{D} calculations (FIG. 29B), the K_{D} ratio of variant compared to unmodified mAb1 was calculated using the equation in FIG. 29C.

Applying this strategy, comparison of K_{D} changes from trastuzumab CDR modifications was derived from AR-SEC-SCX-MS analysis (Table 3). For comparison, the literature reported binding data and potency data on trastuzumab CDR modified variants are also included, showing high consistency with the K_{D} changes calculated using AR-SEC-SCX-MS analysis.

TABLE 3
Comparison of Kp Changes from Trastuzumab CDR Modifications Derived
from AR-SEC-SCX-MS with Binding/Potency Data in Literature

| | CDR Modifications | | | |
|---|---|---|---|---|
| | | | | HC |
| Results | LC N55 > D | LC N30 > D | HC D102 > isoD | D102 > dehydration |
| K_{D} fold change | 1.3 | 6.5 | 5.6 | 2.5 |
| relative | | | | |
| to unmodified | | | | |
| by AR-SEC-MS | | | | |
| Enrichment fold | 1.2 | 4.1 | 3.8 | 1.5 |
| in unbound | | | | |
| fraction by | | | | |
| competitive | | | | |
| binding-based | | | | |
| SEC fractionation | | | | |
| and bottom-up | | | | |
| analysis, | | | | |
| Shi, et al, 2021^{c} | | | | |
| Cell-based | N.A. | 70%^{b} | 9-21%^{b} | N.A. |
| potency testing, | | | | |
| Harris, et al, 2001^{a} | | | | |
| Her2 binding by | N.A. | 58% for two arm | 95% for one | N.A. |
| SPR measurement, | | | | |
| Schmid, et al, 2018^{d} | | deamidated; | arm isomerized. | |
| | | 83% for one arm | | |
| | | deamidated. | | |
| K_{D} fold change | N.A. | 3.9^{f} | 1.2^{f} | |
| relative to | | | | |
| main by SPR | | | | |
| Dakshinamurthy, | | | | |
| et al, 2017^{e} | | | | |

^{a}J Chromatogr B Biomed Sci Appl. 2001 Mar 10;752(2):233-45. doi: 10.1016/s0378-4347(00)00548-x
^{b}potency relative to main peak (main peak = 100%)
^{c}MAbs. 2021 Jan-Dec;13(1):1887612. doi: 10.1080/19420862.2021.1887612.
^{d}Commun Biol. 1, 28, 2018, doi.org/10.1038/s42003-018-0032-8
^{e}Biologicals. 2017 Mar:46:46-56. doi: 10.1016/j.biologicals.2016.12.006
^{f}Analyses were done on Trastuzumab biosimilar mAb

### EXPERIMENTAL

Sample Preparation. To prepare the antigen and antibody mixtures, mAb1 and trastuzumab samples were first subjected to site-specific digestion with FabRICATOR (1 IUB milliunit per 1 μg of protein) in 50 mM Tris-HCl (pH 7.5) at 37° C. for 1 hour, to generate the F(ab′)₂ and Fc fragments. The digestion products were further subjected to limited reduction by incubating with 5 mM DTT in 50 mM Tris-HCl (pH 7.5) at 37° C. for 30 min to selectively reduce inter-chain disulfide bonds, followed by alkylation with 20 mM IAA at room temperature in the dark for 30 min. The FabRICATOR (IdeS) digested and partially reduced sample of mAb1 was mixed with Ag1 at Ag-to-Fab molar ratios of 1:4, 3:8, 5:8, 3:4, and 1:1, respectively. The FabRICATOR (IdeS) digested and partially reduced sample of trastuzumab was mixed with Her2 (ectodomain) at Ag-to-Fab molar ratios of 1:5, 2:5, 3:5, and 5:5, respectively. The total protein concentrations for all mixtures were in the range of 1-5 mg/mL and 2-10 μg of each mixture sample was subjected to analysis.

Affinity-Resolved SEC-SCX-MS. The mixtures containing pre-treated mAb and its binding partner at varying molar ratios were subjected to 2D SEC-SCX-MS analysis as described above. Native SEC chromatography was performed on an Acquity BEH200 SEC column (4.6×150 or 4.6×300 mm, 1.7 μm, 200 Å; Waters, Milford, MA) with the column placed in room temperature. An isocratic flow of 50-75 mM ammonium acetate at 0.2 mL/min was applied to elute and separate the bound complexes from the unbound mAb in the mixtures. For native SCX analysis of the SEC eluted unbound protein fraction, a BioPro IEX SF column (4.6 mm×100 mm, 5 μm; YMC Co., LTD., Kyoto, Japan) was used at 45° C. with a linear gradient of 20 mM ammonium acetate (pH 6.8) to 150 mM ammonium acetate (pH 6.8) for 8 minutes at 0.25 mL/min. The post-column denaturation (PCD) was enabled using a denaturing flow consisting of 60% ACN, 36% water, and 4% FA, which was mixed with the SEC or SCX flow at 1:1 ratio before MS analysis.

### EMBODIMENTS

The following list of embodiments is intended to complement, rather than displace or supersede, the previous descriptions.

### Embodiments Set I

Embodiment I-1. A method for identifying the binding of a peptide or protein to a binding partner, comprising:
contacting a sample comprising the peptide or protein with a binding partner to form a complex;
contacting the complex to a liquid chromatography column to separate bound and free material in an eluate;
contacting the eluate to a denaturation solvent to form a denatured sample;
contacting the denatured sample to a mass spectrometer that is coupled to the liquid chromatography column to determine a mass of the bound and free material; and
analyzing the mass of the bound and free material to identify the binding of the peptide or protein to the binding partner.

Embodiment I-2. The method of embodiment 1, wherein the peptide or protein is digested prior to step (a).

Embodiment I-3. The method of embodiment 2, wherein said digestive enzyme is selected from a group consisting of pepsin, trypsin, Tryp-N, chymotrypsin, Lys-N, Lys-C, Asp-N, Arg-C, Glu-C, papain, IdeS, and variants or combinations thereof.

Embodiment I-4. The method of embodiment 3, wherein said digestive enzyme is IdeS.

Embodiment I-5. The method of embodiment 2, wherein the digested sample is partially reduced to form a digested and partially reduced sample.

Embodiment I-6. The method of embodiment 5, wherein said reducing agent comprises dithiothreitol (DTT), ß-mercaptoethanol, Ellman's reagent, hydroxylamine hydrochloride, sodium cyanoborohydride, tris(2-carboxyethyl)phosphine hydrochloride (TCEP-HCl), or combinations thereof.

Embodiment I-7. The method of embodiment 1, wherein the binding partner is a receptor to the peptide or protein.

Embodiment I-8. The method of embodiment 7, wherein the ratio of receptor to peptide or protein is between 1:10 and 10:1.

Embodiment I-9. The method of embodiment 8, wherein the ratio of receptor to peptide or protein is 1:4, 1:2, 1:1, 2:1, or 4:1.

Embodiment I-10. The method of embodiment 1, wherein the binding partner is an antigen to the peptide or protein.

Embodiment I-11. The method of embodiment 10, wherein the ratio of the antigen to peptide or protein is between 1:10 and 10:1.

Embodiment I-12. The method of embodiment 11, wherein the ratio of antigen to peptide or protein is 1:2, 2:3, 1:1, 2:1, 3:2, 4:1, or 5:2.

Embodiment I-13. The method of embodiment 1, wherein said protein of interest is selected from a group comprising an antibody, a monoclonal antibody, a bispecific antibody, an antibody fragment, an antibody-derived protein, an antigen-binding protein, an antibody-drug conjugate, or a fusion protein.

Embodiment I-14. The method of embodiment 1, wherein said liquid chromatography comprises reversed phase liquid chromatography, ion exchange chromatography, anion exchange chromatography, weak cation exchange chromatography, strong cation exchange chromatography, size exclusion chromatography, affinity chromatography, hydrophobic interaction chromatography, hydrophilic interaction liquid chromatography (HILIC), mixed-mode chromatography, or a combination thereof.

Embodiment I-15. The method of embodiment 14, wherein the liquid chromatography comprises size exclusion chromatography.

Embodiment I-16. The method of embodiment 1, wherein the mobile phase of the liquid chromatography comprises ammonium acetate.

Embodiment I-17. The method of embodiment 1, wherein the denaturation solvent comprises acetonitrile (ACN), water and formic acid (FA).

Embodiment I-18. The method of embodiment 17, wherein the denaturation solvent is added at a 1:1 ratio with the eluent.

Embodiment I-19. The method of embodiment 1, wherein a splitter is used to connect the chromatography column and the mass spectrometer.

Embodiment I-20. The method of embodiment 1, wherein the splitter is used after denaturation to divert a low flow to the mass spectrometer and a high flow to a UV detector.

Embodiment I-21. The method of embodiment 1, wherein the binding partner of the at least one peptide or protein is protein A, protein G, Fc receptor, FcγRIIIa, anti-human Fc antibody, neonatal Fc receptor, Fc epsilon RI, anti-idiotype antibody, an antigen or complement component C1q.

Embodiment I-22. The method of embodiment 1, wherein the mass spectrometer is an electrospray ionization mass spectrometer, nano-electrospray ionization mass spectrometer, a triple quadrupole mass spectrometer, a quadrupole mass spectrometer, or an ultra-high mass range hybrid quadrupole mass spectrometer.

Embodiment I-23. The method of embodiment 22, wherein the mass spectrometer comprises a nano-electrospray ionization-mass spectrometer.

Embodiment I-24. A method for identifying or characterizing the effect of glycosylation on the binding of a peptide or protein to a binding partner, comprising:
contacting a sample comprising the peptide or protein with a binding partner to form a complex;
contacting the complex to a liquid chromatography column to separate bound and free material in an eluate;
contacting the eluate to a denaturation solvent to form a denatured sample;
contacting the denatured sample to a mass spectrometer that is coupled to the liquid chromatography column, to determine a mass of the bound and free material; and
analyzing the mass of the bound and free material to identify or characterize the binding of the peptide or protein to the binding partner.

Embodiment I-25. The method of embodiment 24, wherein the peptide or protein is digested prior to step (a).

Embodiment I-26. The method of embodiment 25, wherein said digestive enzyme is selected from a group consisting of pepsin, trypsin, Tryp-N, chymotrypsin, Lys-N, Lys-C, Asp-N, Arg-C, Glu-C, papain, IdeS, and variants or combinations thereof.

Embodiment I-27. The method of embodiment 26, wherein said digestive enzyme is IdeS.

Embodiment I-28. The method of embodiment 25, wherein the digested sample is partially reduced to form a digested and partially reduced sample.

Embodiment I-29. The method of embodiment 28, wherein said reducing agent comprises dithiothreitol (DTT), ß-mercaptoethanol, Ellman's reagent, hydroxylamine hydrochloride, sodium cyanoborohydride, tris(2-carboxyethyl)phosphine hydrochloride (TCEP-HCl), or combinations thereof.

Embodiment I-30. The method of embodiment 24, wherein the binding partner is a receptor to the peptide or protein.

Embodiment I-31. The method of embodiment 30, wherein the ratio of receptor to peptide or protein is between 1:10 and 10:1.

Embodiment I-32. The method of embodiment 31, wherein the ratio of receptor to peptide or protein is 1:4, 1:2, 1:1, 2:1, or 4:1.

Embodiment I-33. The method of embodiment 24, wherein the binding partner is an antigen to the peptide or protein.

Embodiment I-34. The method of embodiment 33, wherein the ratio of the antigen to peptide or protein is between 1:10 and 10:1.

Embodiment I-35. The method of embodiment 34, wherein the ratio of antigen to peptide or protein is 1:2, 2:3, 1:1, 2:1, 3:2, 4:1, or 5:2.

Embodiment I-36. The method of embodiment 24, wherein said protein of interest is selected from a group comprising an antibody, a monoclonal antibody, a bispecific antibody, an antibody fragment, an antibody-derived protein, an antigen-binding protein, an antibody-drug conjugate, or a fusion protein.

Embodiment I-37. The method of embodiment 24, wherein said liquid chromatography comprises reversed phase liquid chromatography, ion exchange chromatography, anion exchange chromatography, weak cation exchange chromatography, strong cation exchange chromatography, size exclusion chromatography, affinity chromatography, hydrophobic interaction chromatography, hydrophilic interaction liquid chromatography (HILIC), mixed-mode chromatography, or a combination thereof.

Embodiment I-38. The method of embodiment 37, wherein the liquid chromatography comprises size exclusion chromatography.

Embodiment I-39. The method of embodiment 24, wherein the mobile phase of the liquid chromatography comprises ammonium acetate.

Embodiment I-40. The method of embodiment 24, wherein the denaturation solvent comprises acetonitrile (ACN), water and formic acid (FA).

Embodiment I-41. The method of embodiment 40, wherein the denaturation solvent is added at a 1:1 ratio with the eluent.

Embodiment I-42. The method of embodiment 24, wherein a splitter is used to connect the chromatography column and the mass spectrometer.

Embodiment I-43. The method of embodiment 24, wherein the splitter is used after denaturation to divert a low flow to the mass spectrometer and a high flow to a UV detector.

Embodiment I-44. The method of embodiment 24, wherein the binding partner of the at least one peptide or protein is protein A, protein G, Fcγ receptor, FcγRIIIa, anti-human Fc antibody, neonatal Fc receptor, Fc epsilon RI, anti-idiotype antibody, an antigen or complement component C1q.

Embodiment I-45. The method of embodiment 24, wherein the mass spectrometer is an electrospray ionization mass spectrometer, nano-electrospray ionization mass spectrometer, a triple quadrupole mass spectrometer, a quadrupole mass spectrometer or an ultra-high mass range hybrid quadrupole mass spectrometer.

Embodiment I-46. The method of embodiment 45, wherein the mass spectrometer comprises a nano-electrospray ionization-mass spectrometer.

Embodiment I-47. A method for identifying or characterizing the effect of an Fc region variant on the binding of a peptide or protein to a binding partner, comprising:
contacting a sample comprising the peptide or protein with a binding partner to form a complex;
contacting the complex to a liquid chromatography column to separate bound and free material in an eluate;
contacting the eluate to a denaturation solvent to form a denatured sample;
contacting the denatured sample to a mass spectrometer that is coupled to the liquid chromatography column, to determine a mass of the bound and free material; and
analyzing the mass of the bound and free material to identify or characterize the binding of the peptide or protein to the binding partner.

Embodiment I-48. The method of embodiment 47, wherein the binding partner is a receptor to the peptide or protein.

Embodiment I-49. The method of embodiment 47, wherein the ratio of receptor to peptide or protein is between 1:10 and 10:1.

Embodiment I-50. The method of embodiment 49, wherein the ratio of receptor to peptide or protein is 1:6, 1:3, 2:3, 4:3, and 6:3.

Embodiment I-51. The method of embodiment 47, wherein said protein of interest is selected from a group comprising an antibody, a monoclonal antibody, a bispecific antibody, an antibody fragment, an antibody-derived protein, an antigen-binding protein, an antibody-drug conjugate, or a fusion protein.

Embodiment I-52. The method of embodiment 47, wherein said liquid chromatography comprises reversed phase liquid chromatography, ion exchange chromatography, anion exchange chromatography, weak cation exchange chromatography, strong cation exchange chromatography, size exclusion chromatography, affinity chromatography, hydrophobic interaction chromatography, hydrophilic interaction liquid chromatography (HILIC), mixed-mode chromatography, or a combination thereof.

Embodiment I-53. The method of embodiment 52, wherein the liquid chromatography comprises size exclusion chromatography.

Embodiment I-54. The method of embodiment 47, wherein the mobile phase of the liquid chromatography comprises ammonium acetate.

Embodiment I-55. The method of embodiment 47, wherein the denaturation solvent comprises acetonitrile (ACN), and water.

Embodiment I-56. The method of embodiment 55, wherein the denaturation solvent is added at a 1:1 ratio with the eluent.

Embodiment I-57. The method of embodiment 47, wherein a splitter is used to connect the chromatography column and the mass spectrometer.

Embodiment I-58. The method of embodiment 47, wherein the splitter is used after denaturation to divert a low flow to the mass spectrometer and a high flow to a UV detector.

Embodiment I-59. The method of embodiment 47, wherein the binding partner of the at least one peptide or protein is protein A, protein G, Fcγ receptor, FcγRIIIa, anti-human Fc antibody, neonatal Fc receptor, Fc epsilon RI, anti-idiotype antibody, an antigen or complement component C1q.

Embodiment I-60. The method of embodiment 47, wherein the mass spectrometer is an electrospray ionization mass spectrometer, nano-electrospray ionization mass spectrometer, a triple quadrupole mass spectrometer, a quadrupole mass spectrometer or an ultra-high mass range hybrid quadrupole mass spectrometer.

Embodiment I-61. The method of embodiment 60, wherein the mass spectrometer comprises a nano-electrospray ionization-mass spectrometer.

### Embodiments Set II

Embodiment II-1. A method of characterizing the binding of a protein to an antigen, the method comprising:
incubating the antigen with a sample comprising the protein to form a mixture comprising protein-antigen complex and protein that is not bound to the antigen;
separating the mixture on a liquid chromatography column to separate the protein-antigen complex from the protein that is not bound to the antigen and to form an eluate comprising the protein-antigen complex and the protein that is not bound to the antigen;
contacting the eluate with a denaturation solvent to form a denatured sample;
determining a mass of the protein-antigen complex and a mass of the protein that is not bound to the antigen with a mass spectrometer that is coupled to the liquid chromatography column; and
analyzing the mass of the protein-antigen complex and the mass of the protein that is not bound to the antigen to characterize the binding of the protein to the antigen.

Embodiment II-2. The method of embodiment 1, wherein the protein is digested by pepsin, trypsin, Tryp-N, chymotrypsin, Lys-N, Lys-C, Asp-N, Arg-C, Glu-C, papain, IdeS, and variants or combinations thereof prior to the incubating step.

Embodiment II-3. The method of embodiment 2, wherein the protein is digested with IdeS prior to the incubating step.

Embodiment II-4. The method of embodiment 1, wherein the protein is reduced dithiothreitol (DTT), ß-mercaptoethanol, Ellman's reagent, hydroxylamine hydrochloride, sodium cyanoborohydride, tris(2-carboxyethyl)phosphine hydrochloride (TCEP-HCl), or combinations thereof.

Embodiment II-5. The method of embodiment 1, wherein the ratio of antigen to protein in the incubating step is between 1:10 and 10:1.

Embodiment II-6. The method of embodiment 1, wherein the protein is selected from an antibody, a monoclonal antibody, a bispecific antibody, an antibody fragment, an antibody-derived protein, an antigen-binding protein, an antibody-drug conjugate, or a fusion protein.

Embodiment II-7. The method of embodiment 1, wherein the liquid chromatography comprises reversed phase liquid chromatography, ion exchange chromatography, anion exchange chromatography, weak cation exchange chromatography, strong cation exchange chromatography, size exclusion chromatography, affinity chromatography, hydrophobic interaction chromatography, hydrophilic interaction liquid chromatography (HILIC), mixed-mode chromatography, or a combination thereof.

Embodiment II-8. The method of embodiment 1, wherein the mobile phase of the liquid chromatography comprises ammonium acetate.

Embodiment II-9. The method of embodiment 1, wherein the denaturation solvent comprises acetonitrile (ACN), water, and formic acid (FA).

Embodiment II-10. The method of embodiment 9, wherein the denaturation solvent is added at a 1:1 ratio with the eluent.

Embodiment II-11. The method of embodiment 1, wherein the antigen is protein A, protein G, Fcγ receptor, FcγRIIIa, anti-human Fe antibody, neonatal Fc receptor, Fe epsilon RI, anti-idiotype antibody, or complement component C1q.

Embodiment II-12. The method of embodiment 1, wherein the liquid chromatography column eluate comprises 50 mM-75 mM ammonium acetate.

Embodiment II-13. The method of embodiment 1, wherein following the analysis of the mass of the protein-antigen complex and the mass of the protein that is not bound to the antigen, the method further comprises:
separating the protein that is not bound to the antigen on a strong cation exchange (SCX) chromatography column to separate modified and unmodified protein and to form an eluate comprising modified protein and unmodified protein;
contacting the eluate comprising the modified protein and the unmodified protein with a denaturation solvent to form a denatured sample;
determining a mass of the modified protein and a mass of the unmodified protein with a mass spectrometer that is coupled to the strong cation exchange chromatography column; and
analyzing the mass of the modified protein and the mass of the unmodified protein to determine if the protein that is not bound to the antigen has a molecular modification.

Embodiment II-14. The method of embodiment 13, wherein the strong cation exchange chromatography column separation buffer comprises 20 mM ammonium acetate.

Embodiment II-15. The method of embodiment 13, wherein the molecular modification comprises asparagine deamidation, aspartic acid isomerization, aspartic acid dehydration, or any combination thereof.

Embodiment II-16. The method of embodiment 1, wherein the method is used to characterize the effect of an Fc region variant on the binding of the protein to the antigen.

Embodiment II-17. A method for analyzing the effect of glycosylation on the binding of a protein to an antigen, the method comprising:
incubating the antigen with a sample comprising the protein to form a mixture comprising protein-antigen complex and protein that is not bound to the antigen;
separating the mixture on a liquid chromatography column to separate the protein-antigen complex from the protein that is not bound to the antigen and to form an eluate comprising the protein-antigen complex and the protein that is not bound to the antigen;
contacting the eluate with a denaturation solvent to form a denatured sample;
determining a mass of the protein-antigen complex and a mass of the protein that is not bound to the antigen with a mass spectrometer that is coupled to the liquid chromatography column; and
analyzing the mass of the protein-antigen complex and the mass of the protein that is not bound to the antigen to analyze the effect of glycosylation on the binding of the protein to the antigen.

Embodiment II-18. A method of characterizing the binding of a protein to an antigen, the method comprising:
incubating the antigen with a sample comprising the protein to form a mixture comprising protein-antigen complex and protein that is not bound to the antigen;
separating the mixture on a liquid chromatography column to separate the protein-antigen complex from the protein that is not bound to the antigen and to form an eluate comprising the protein-antigen complex and the protein that is not bound to the antigen;
contacting the eluate with a denaturation solvent to form a denatured sample;
determining a mass of the protein-antigen complex and a mass of the protein that is not bound to the antigen with a mass spectrometer that is coupled to the liquid chromatography column;
analyzing the mass of the protein-antigen complex and the mass of the protein that is not bound to the antigen to characterize the binding of the protein to the antigen;
separating the protein that is not bound to the antigen on a strong cation exchange (SCX) chromatography column to separate modified protein and unmodified protein and to form an eluate comprising modified and unmodified protein;
contacting the eluate comprising the modified protein and the unmodified protein with a denaturation solvent to form a denatured sample;
determining a mass of the modified protein and a mass of the unmodified protein with a mass spectrometer that is coupled to the strong cation exchange chromatography column; and
analyzing the mass of the modified protein and the mass of the unmodified protein to determine if the protein that is not bound to the antigen has a molecular modification.

Embodiment II-19. The method of embodiment 18, wherein the molecular modification comprises asparagine deamidation, aspartic acid isomerization, aspartic acid dehydration, or any combination thereof.

What is claimed is:

1. A method of characterizing the binding of a protein to an antigen, the method comprising:
incubating the antigen with a sample comprising the protein to form a mixture comprising protein-antigen complex and protein that is not bound to the antigen;
separating the mixture on a liquid chromatography column to separate the protein-antigen complex from the protein that is not bound to the antigen and to form an eluate comprising the protein-antigen complex and the protein that is not bound to the antigen;
contacting the eluate with a denaturation solvent to form a denatured sample;
determining a mass of the protein-antigen complex and a mass of the protein that is not bound to the antigen with a mass spectrometer that is coupled to the liquid chromatography column; and
analyzing the mass of the protein-antigen complex and the mass of the protein that is not bound to the antigen to characterize the binding of the protein to the antigen.

2. The method of claim 1, wherein the protein is digested by pepsin, trypsin, Tryp-N, chymotrypsin, Lys-N, Lys-C, Asp-N, Arg-C, Glu-C, papain, IdeS, and variants or combinations thereof prior to the incubating step.

3. The method of claim 2, wherein the protein is digested with IdeS prior to the incubating step.

4. The method of claim 1, wherein the protein is reduced dithiothreitol (DTT), ß-mercaptoethanol, Ellman's reagent, hydroxylamine hydrochloride, sodium cyanoborohydride, tris(2-carboxyethyl)phosphine hydrochloride (TCEP-HCl), or combinations thereof.

5. The method of claim 1, wherein the ratio of antigen to protein in the incubating step is between 1:10 and 10:1.

6. The method of claim 1, wherein the protein is selected from an antibody, a monoclonal antibody, a bispecific antibody, an antibody fragment, an antibody-derived protein, an antigen-binding protein, an antibody-drug conjugate, or a fusion protein.

7. The method of claim 1, wherein the liquid chromatography comprises reversed phase liquid chromatography, ion exchange chromatography, anion exchange chromatography, weak cation exchange chromatography, strong cation exchange chromatography, size exclusion chromatography, affinity chromatography, hydrophobic interaction chromatography, hydrophilic interaction liquid chromatography (HILIC), mixed-mode chromatography, or a combination thereof.

8. The method of claim 1, wherein the mobile phase of the liquid chromatography comprises ammonium acetate.

9. The method of claim 1, wherein the denaturation solvent comprises acetonitrile (ACN), water, and formic acid (FA).

10. The method of claim 9, wherein the denaturation solvent is added at a 1:1 ratio with the eluent.

11. The method of claim 1, wherein the antigen is protein A, protein G, Fcγ receptor, FcγRIIIa, anti-human Fc antibody, neonatal Fc receptor, Fc epsilon RI, anti-idiotype antibody, or complement component C1q.

12. The method of claim 1, wherein the liquid chromatography column eluate comprises 50 mM-75 mM ammonium acetate.

13. The method of claim 1, wherein following the analysis of the mass of the protein-antigen complex and the mass of the protein that is not bound to the antigen, the method further comprises:
separating the protein that is not bound to the antigen on a strong cation exchange (SCX) chromatography column to separate modified and unmodified protein and to form an eluate comprising modified protein and unmodified protein;
contacting the eluate comprising the modified protein and the unmodified protein with a denaturation solvent to form a denatured sample;
determining a mass of the modified protein and a mass of the unmodified protein with a mass spectrometer that is coupled to the strong cation exchange chromatography column; and
analyzing the mass of the modified protein and the mass of the unmodified protein to determine if the protein that is not bound to the antigen has a molecular modification.

14. The method of claim 13, wherein the strong cation exchange chromatography column separation buffer comprises 20 mM ammonium acetate.

15. The method of claim 13, wherein the molecular modification comprises asparagine deamidation, aspartic acid isomerization, aspartic acid dehydration, or any combination thereof.

16. The method of claim 1, wherein the method is used to characterize the effect of an Fc region variant on the binding of the protein to the antigen.

17. A method for analyzing the effect of glycosylation on the binding of a protein to an antigen, the method comprising:
incubating the antigen with a sample comprising the protein to form a mixture comprising protein-antigen complex and protein that is not bound to the antigen;
separating the mixture on a liquid chromatography column to separate the protein-antigen complex from the protein that is not bound to the antigen and to form an eluate comprising the protein-antigen complex and the protein that is not bound to the antigen;
contacting the eluate with a denaturation solvent to form a denatured sample;
determining a mass of the protein-antigen complex and a mass of the protein that is not bound to the antigen with a mass spectrometer that is coupled to the liquid chromatography column; and
analyzing the mass of the protein-antigen complex and the mass of the protein that is not bound to the antigen to analyze the effect of glycosylation on the binding of the protein to the antigen.

18. A method of characterizing the binding of a protein to an antigen, the method comprising:
incubating the antigen with a sample comprising the protein to form a mixture comprising protein-antigen complex and protein that is not bound to the antigen;
separating the mixture on a liquid chromatography column to separate the protein-antigen complex from the protein that is not bound to the antigen and to form an eluate comprising the protein-antigen complex and the protein that is not bound to the antigen;
contacting the eluate with a denaturation solvent to form a denatured sample;
determining a mass of the protein-antigen complex and a mass of the protein that is not bound to the antigen with a mass spectrometer that is coupled to the liquid chromatography column;
analyzing the mass of the protein-antigen complex and the mass of the protein that is not bound to the antigen to characterize the binding of the protein to the antigen;
separating the protein that is not bound to the antigen on a strong cation exchange (SCX) chromatography column to separate modified protein and unmodified protein and to form an eluate comprising modified and unmodified protein;
contacting the eluate comprising the modified protein and the unmodified protein with a denaturation solvent to form a denatured sample;
determining a mass of the modified protein and a mass of the unmodified protein with a mass spectrometer that is coupled to the strong cation exchange chromatography column; and
analyzing the mass of the modified protein and the mass of the unmodified protein to determine if the protein that is not bound to the antigen has a molecular modification.

19. The method of claim 18, wherein the molecular modification comprises asparagine deamidation, aspartic acid isomerization, aspartic acid dehydration, or any combination thereof.
